SEQUENCE LISTING (21 sequences)

SEQ ID NO: 1 - protein, 41 aa, Homo sapiens
GIGDPVTCLKSGAICHPVFCPRRYKQIGTCGLPGTKCCKKP

SEQ ID NO: 2 - protein, 64 aa, Homo sapiens
MRVLYLLFSFLFIFLMPLPGVFGGIGDPVTCLKSGAICHPVFCPRRYKQIGTCGLPGTKCCKKP

SEQ ID NO: 3 - protein, 121 aa, Fusobacterium nucleatum
MSLFLVACGEKKEEEKPAEQAAVEATATEAPATETTEAAAEAKTFSLKTEDGKEFTLVVAADGSTATLTDAEGKATELKNAETASGERYADEAGNEVAMKGAEGILTLGDLKEVPVTVEAK

SEQ ID NO: 4 - DNA, 2045 bp, Homo sapiens
ctttataaggtggaaggcttgatgtcctccccagactcagctcctggtgaagctcccagccatcagccatgagggtcttgtatctcctcttctcgttcctcttcatattcctgatgcctcttccaggtgagatgggccagggaaataggagggttggccaaatggaagaatggcgtagaagttctctgtctcctctcattcccctccacctatctctccctcatccctctctctccttcctctctctgtgtgtcccctccatccttttctcctgcttctctctcttcttccctctctctcttttttctgtctttctttttcctctctccctagagcatgtctttctttctttctctttcctttcttctacccacacttttagactgaatgccctatttaattgaacaaagcattgcttccttcaatagaaaaggagtttgagaacccaatggacacctcactcgttcttctaagccaatatgaaggagcccagtagcttgtaaatatcatctcttcactgctttccatgctacaactgctgagactatggttgaaacctgttaggtgactttttaaataaaaggcagaaattttgattttatctaaagaaagtagtatagaatgtcattttctaaatttttatatttaaagggtagatactgcaacctagagaattccagataatcttaaggcccagcctatactgtgagaactactgcagcaagacactctgcctccaggacttttctgatcagaggccctgagaacagtccctgccactaggccactgcaggttcacaggacagggtacagcccattgaaacctacttttaaacctggatgcctaaccttcattttctccttgatattatgaaaataaaataaaaaccatgaaaggataaaagagggagagtggaagggaaggatggagaaagggaaaaagaaaatttgagagtaaatcctaaaacaattaatctaatagatatcatcttgtgaaatcctcattttaccaatcttatttatgagtcctgggttttgtgagaacaatggggttctgagaggcaccagagacctcatgttttccaaaacctagaacagtataatgaaggaaggcggggaggcagggaggcagggaggcagggaggcagggaggcgggcaggtggggagggagggacggaaggagggagggagggagggagggagggagggagggataaaaaaagaagaatgaggttgaaaccaggacttagatattagaaacaagccattacaaaatttatttctatggttaattgtggttttcaactgtaagttacttggtgttaatttcctattaaacaatttcagtaagttgcatctttttatcccatctcaggtcaaatacttaacagactaaatgatttgaaaaagcaaaagtttactggcttgtgtgtgttaaaatggaggtatggtggctttgatattatcttcttgtggtggagctgaattcacaagagatcgttgctgagctcctaccagaccccacctggaggccccagtcactcaggagagatcagggtctttcacaatcaggttctacaaaaataaacatccccccaaccacagcagtgccagtttccatgtcagaaacttagatccaaatgactgactcgcgtctcattatcatgatggaaaagcccaggcttgagaaagaagcccgctgcggatttactcaaggcgatactgacacagggtttgtgtttttccaacatgagttttgagttcttacacgctgtttgctctttttgtgtgttttttccctgttaggtgtttttggtggtataggcgatcctgttacctgccttaagagtggagccatatgtcatccagtcttttgccctagaaggtataaacaaattggcacctgtggtctccctggaacaaaatgctgcaaaaagccatgaggaggccaagaagctgctgtggctgatgcggattcagaaagggctccctcatcagagacgtgcgacatgtaaaccaaattaaactatggtgtccaaagata

SEQ ID NO: 5 - DNA, 319 bp, Homo sapiens
ggtgaagctcccagccatcagccatgagggtcttgtatctcctcttctcgttcctcttcatattcctgatgcctcttccaggtgtttttggtggtataggcgatcctgttacctgccttaagagtggagccatatgtcatccagtcttttgccctagaaggtataaacaaattggcacctgtggtctccctggaacaaaatgctgcaaaaagccatgaggaggccaagaagctgctgtggctgatgcggattcagaaagggctccctcatcagagacgtgcgacatgtaaaccaaattaaactatggtgtccaaagata

SEQ ID NO: 6 - DNA, 195 bp, Homo sapiens
atgagggtcttgtatctcctcttctcgttcctcttcatattcctgatgcctcttccaggtgtttttggtggtataggcgatcctgttacctgccttaagagtggagccatatgtcatccagtcttttgccctagaaggtataaacaaattggcacctgtggtctccctggaacaaaatgctgcaaaaagccatga

SEQ ID NO: 7 - DNA, 126 bp, Homo sapiens
ggtataggcgatcctgttacctgccttaagagtggagccatatgtcatccagtcttttgccctagaaggtataaacaaattggcacctgtggtctccctggaacaaaatgctgcaaaaagccatga

SEQ ID NO: 8 - DNA, 366 bp, Homo sapiens
atgagtttattcttagtagcttgtggagaaaaaaaagaagaagaaaaaccagctgaacaagctgctgtagaagcaactgcaactgaagcacctgctacagaaacaactgaagctgctgctgaagctaaaacattctcacttaaaactgaagatggaaaagaattcacattagtagttgctgctgatggaagtactgcaactttaactgatgcagaaggaaaagcaactgaattaaaaaatgctgaaactgcatctggagaaagatatgcagatgaagctggaaacgaagttgctatgaaaggtgcagaaggaatcttaactttaggagaccttaaagaagtaccagtaactgttgaagctaaatag

SEQ ID NO: 9 - protein, 129 aa, Fusobacterium nucleatum
MKKILLLLSSLFLFACANIDTGVDESKEAQISRLLKEADKKKEKTVEVEKKLVTDNGEEVIEEEATVQNKKSHKGMTRGEIMEYEMTRVSDEMNALQADVQQYQEKKAQLKAYQEKLQKLEELNNAGIK

SEQ ID NO: 10 - DNA, 390 bp, Fusobacterium nucleatum
ttgaaaaaaatattattactattatcttctttatttttatttgcttgtgctaatatagatacaggtgtagatgaaagtaaagaagctcaaatatcaagacttttaaaagaagctgataagaaaaaagaaaaaacagtagaagtagaaaagaaacttgtaactgataatggagaggaagttatagaggaagaagctaccgttcaaaacaaaaaatcacataaaggaatgacaagaggggaaataatggaatatgaaatgacaagagtttcagatgaaatgaatgccctacaagcggatgtacaacaatatcaagaaaagaaagcacaactaaaagcataccaagaaaaattacaaaaattagaagaattaaataatgcaggaataaaataa

SEQ ID NO: 11 - protein, 123 aa, Fusobacterium nucleatum
MKKVILTLFVLLSIGIFANDEIISELKGLNAEYENLVKEEEARFQKEKELSERAAAQNVKLAELKASIEEKLLAAPEERKTKFFKDTFDGLVKDYSKYLSQINEKIAENTEIVSNFEKIQKIR

SEQ ID NO: 12 - DNA, 372 bp, Fusobacterium nucleatum
atgaaaaaagttattttaacattatttgttttattatctattggaatatttgcaaatgatgagattatttcagagttaaaaggacttaatgctgagtatgaaaatttagtaaaagaagaagaagctagatttcaaaaagaaaaagaactttctgaaagagcagcagctcaaaatgttaaattggctgaattaaaagcaagcattgaagaaaaattgttagcagctccagaagaaagaaaaacaaaattttttaaagatacttttgatggtttagtgaaagattattcaaaatatttaagtcaaataaatgaaaaaatagctgaaaatactgaaatagtaagtaattttgaaaaaattcaaaaaataagatag

SEQ ID NO: 13 - protein, 129 aa, Fusobacterium nucleatum
MKKFLLLAVLAVSASAFAANDAASLVGELQALDAEYQNLANQEEARFNEERAQADAARQALAQNEQVYNELSQRAQRLQAEANTRFYKSQYQDLASKYEDALKKLESEMEQQKAIISDFEKIQALRAGN

SEQ ID NO: 14 - DNA, 390 bp, Fusobacterium nucleatum
atgaaaaaatttttattattagcagtattagctgtttctgcttcagcattcgcagcaaatgatgcagcaagtttagtaggtgaattacaagcattagatgctgaataccaaaacttagcaaatcaagaagaagcaagattcaatgaagaaagagcacaagctgacgctgctagacaagcactagcacaaaatgaacaagtttacaatgaattatctcaaagagctcaaagacttcaagctgaagctaacacaagattttataaatctcaataccaagatctagcttctaaatatgaagacgctttaaagaaattagaatctgaaatggaacaacaaaaagctattatttctgattttgaaaaaattcaagctttaagagctggtaactaa

SEQ ID NO: 15 - protein, 67 aa, Homo sapiens
MRIHYLLFALLFLFLVPVPGHGGIINTLQKYYCRVRGGRCAVLSCLPKEEQIGKCSTRGRKCCRRKK

SEQ ID NO: 16 - DNA, 204 bp, Homo sapiens
atgaggatccattatcttctgtttgctttgctcttcctgtttttggtgcctgttccaggtcatggaggaatcataaacacattacagaaatattattgcagagtcagaggcggccggtgtgctgtgctcagctgccttccaaaggaggaacagatcggcaagtgctcgacgcgtggccgaaaatgctgccgaagaaagaaataa

SEQ ID NO: 17 - DNA, 337 bp, Homo sapiens
tgagtctcagcgtggggtgaagcctagcagctatgaggatccattatcttctgtttgctttgctcttcctgtttttggtgcctgtcccaggtcatggaggaatcataaacacattacagaaatattattgcagagtcagaggcggccggtgtgctgtgctcagctgccttccaaaggaggaacagatcggcaagtgctcgacgcgtggccgaaaatgctgccgaagaaagaaataaaaaccctgaaacatgacgagagtgttgtaaagtgtggaaatgccttcttaaagtttataaaagtaaaatcaaattacatttttttttcaaaaaaaaaaaaa

SEQ ID NO: 18 - DNA, 266 bp, Homo sapiens
catccagtctcagcgtggggtgaagcctagcagctatgaggatccattatcttctgtttgctttgctcttcctgtttttggtgcctgttccaggtcatggaggaatcataaacacattacagaaatattattgcagagtcagaggcggccggtgtgctgtgctcagctgccttccaaaggaggaacagatcggcaagtgctcgacgcgtggccgaaaatgctgccgaagaaagaaataaaaaccctgaaacatgacgagagtgttg

SEQ ID NO: 19 - protein, 82 aa, Homo sapiens
  VARIANT 81: Xaa = Any Amino Acid
SLWDQSLKPCVKLTPLCVTLNCRDVNATNTGNVTYNDTIKGEIKNCSFNTTTEIRDKKQTAYALFYKLDIVPLNDGNNNNXY

SEQ ID NO: 20 - protein, 64 aa, Homo sapiens
  VARIANT 21,23,36: Xaa = Any Amino Acid
SLWDQSLKPCVKLTPLCVTLXCXNATFNNITTFNIXNSSSNITTYPINNTTNQHSLFYNLHVLP

SEQ ID NO: 21 - protein, 101 aa, Homo sapiens
SLWDQSLKPCVKLTPLCVTLKCENATINNGGNATVASNDTINREVKNCSFNITTDLRDKRKHEYALFYTLDIVPLNEKKNNASEYRLISCNTSAVTQACPK

(19) United States
(12) Patent Application Publication
(10) Pub. No.: US 2004/0224883 A1
(43) Pub. Date: Nov. 11, 2004

(54) Compositions and methods for treating HIV infections

(75) Inventor: Aaron Weinberg, Shaker Heights, OH (US)

Correspondence Address: ROPES & GRAY LLP, ONE INTERNATIONAL PLACE, BOSTON, MA, 02110-2624, US

(73) Assignee: Case Western Reserve University, Cleveland, OH (US)

(21) Appl. No.: 10/737,288

(22) Filed: Dec. 15, 2003

Related U.S. Application Data
(60) Provisional application No. 60/433,099, filed on Dec. 13, 2002.

(51) Int. Cl. A61K 38/17

(52) U.S. Cl. 514/12

(57) ABSTRACT

The application provides, in part, compositions and methods for the treatment or on of HIV and other viruses associated with certain chemokine receptors.

### FUNDING

Work described herein was funded, in part, by NIH grants RO-1 DE12589, RO-1 DE13992, and RO-1 DE015510. The United States government has certain rights in the invention.

### BACKGROUND

Fully satisfactory treatments for Human Immunodeficiency Virus (HIV) have not yet been discovered. Mixtures of agents that target the reverse transcriptase and the protease have proven to be highly effective. However, patients are forced to self-administer a large number of medications on a tightly regulated schedule. Failure to follow the prescribed regimen results in rapid generation of drug-resistant HIV mutants. Antiviral agents taken individually are ineffective, largely because of the rapid rate at which the infecting virus population becomes resistant. For this reason, single and multiple drug therapies are often denied to patients that seem unlikely to be able to follow the required dosing schedule. In addition, many protease inhibitors are expensive to manufacture and are not widely available in regions where HIV is rampant, including sub-Saharan Africa and South-East Asia.

The present application provides novel agents for the treatment of HIV and other viral infections associated with certain chemokine receptors, e.g., CXCR4.

### BRIEF SUMMARY

In certain aspects, the application relates to compositions and methods for inhibiting an infection by HIV or a virus associated with certain chemokine receptors, such as for example the CXCR4 receptor by administering to the subject an effective amount of an agent selected from the group consisting of: a beta defensin agent (BD agent) or a beta defensin-inducing agent (BD-inducing agent). The BD-agent may be beta-defensin 2 (BD-2) or beta-defensin 3 (BD-3). In one embodiment the BD agent is a human BD agent (HBD) such as for example HBD-2 or HBD-3.

In certain aspects the application relates to a method for inhibiting the contraction of an HIV infection in a subject, the method comprising administering to the subject an effective amount of an agent selected from the group consisting of: an BD agent; and an BD-inducing agent.

In certain aspects the application relates to a method for inhibiting HIV entry into a cell, the method comprising contacting the cell with an effective amount of an agent selected from the group consisting of: an BD agent; and an BD-inducing agent.

As discussed above, an HBD agent may be an HBD-2 agent or an HBD-3 agent. In certain embodiments, the methods and compositions disclosed herein may employ, as an HBD-2 agent, a polypeptide comprising an amino acid sequence at least 80%, 85%, 90%, 95%, 97%, or 98-99% identical to an amino acid sequence selected from the group consisting of: SEQ ID NO:1 and SEQ ID NO:2. In certain embodiments, the HBD-2 agent is a polypeptide obtained by expressing a nucleic acid that is at least 80%, 85%, 90%, 95%, 97%, or 98-99% identical to a nucleic acid selected from the group consisting of: SEQ ID NOs: 4-7 in a cell. In certain embodiments, the HBD-2 agent is a polypeptide encoded by a nucleic acid that is at least 80%, 85%, 90%, 95%, 97%, or 98-99% identical to a nucleic acid selected from the group consisting of: SEQ ID NOs:4-7. In certain embodiments, the HBD agent is a polypeptide of SEQ ID NO: 1-2 or is encoded by a nucleic acid as set forth in SEQ ID Nos: 4-7. Preferably the HBD-2 agent has a 50% effectiveness at a concentration of about 10 micromolar or less.

In certain embodiments, the methods and compositions disclosed herein may employ, as an HBD-3 agent, a polypeptide comprising an amino acid sequence at least 80%, 85%, 90%, 95%, 97%, or 98-99% identical to an amino acid sequence selected from the group consisting of: SEQ ID NO:15. In certain embodiments, the HBD-3 agent is a polypeptide obtained by expressing a nucleic acid that is at least 80%, 85%, 90%, 95%, 97%, or 98-99% identical to a nucleic acid selected from the group consisting of: SEQ ID NOs:16-18 in a cell. In certain embodiments, the HBD-3 agent is a polypeptide encoded by a nucleic acid that is at least 80%, 85%, 90%, 95%, 97%, or 98-99% identical to a nucleic acid selected from the group consisting of: SEQ ID NOs:16-18. In certain embodiments, the HBD agent is a polypeptide of SEQ ID NO: 15 or is encoded by a nucleic acid as set forth in SEQ ID Nos:16-18. Preferably the HBD-3 agent has a 50% effectiveness at a concentration of about 10 micromolar or less.

A BD-inducing agent is an agent that induces a beta defensin such as for example BD-2 or BD-3. In certain embodiments, the BD-inducing agent induces a HBD-2 or HBD-3. In yet other embodiments, the BD-inducing agent induces a polypeptide comprising an amino acid sequence at least 80%, 85%, 90%, 95%, 97%, or 98-99% identical to an amino acid sequence selected from the group consisting of: SEQ ID NO:1 and SEQ ID NO:2. In certain embodiments, the BD-inducing agent induces a polypeptide obtained by expressing a nucleic acid that is at least 80%, 85%, 90%, 95%, 97%, or 98-99% identical to a nucleic acid selected from the group consisting of: SEQ ID NOs: 4-7 in a cell. In certain embodiments, the BD-inducing agent induces a polypeptide encoded by a nucleic acid that is at least 80%, 85%, 90%, 95%, 97%, or 98-99% identical to a nucleic acid selected from the group consisting of: SEQ ID NOs:4-7.

In yet other embodiments, a BD-inducing agent induces a polypeptide comprising an amino acid sequence at least 80%, 85%, 90%, 95%, 97%, or 98-99% identical to an amino acid sequence of: SEQ ID NO:15. In certain embodiments, a BD-inducing agent induces a polypeptide obtained by expressing a nucleic acid that is at least 80%, 85%, 90%, 95%, 97%, or 98-99% identical to a nucleic acid selected from the group consisting of: SEQ ID NOs:16-18 in a cell. In certain embodiments, a BD-inducing agent induces a polypeptide encoded by a nucleic acid that is at least 80%, 85%, 90%, 95%, 97%, or 98-99% identical to a nucleic acid selected from the group consisting of: SEQ ID NOs:16-18.

A BD-inducing agent may be selected from the group consisting of a polypeptide and portions thereof, a fusion protein, a small molecule, a peptidomimetic, and/or a nucleic acid agent.

In one aspect, the invention also provides screening assays to identify candidate agents that may be BD-inducing agents.

In certain embodiments, small molecules are candidate agents to be screened. In certain preferred embodiments, small molecules are generated by combinatorial synthesis.

In certain embodiments, the methods and compositions disclosed herein may employ, as an BD-inducing agent, a FAD-I polypeptide. In certain embodiments, an FAD-I polypeptide is a polypeptide comprising an amino acid sequence at least 80%, 85%, 90%, 95%, 97%, or 98-99% identical to an amino acid sequence of SEQ ID NO: 3, 9, 11, or 13. In certain embodiments, an FAD-I polypeptide is a polypeptide obtained by expressing a nucleic acid that is at least 80%, 85%, 90%, 95%, 97%, or 98-99% identical to a nucleic acid of SEQ ID NO:8, 10, 12, or 14 in a cell. In certain embodiments, a FAD-I polypeptide is a polypeptide encoded by a nucleic acid that is at least 80%, 85%, 90%, 95%, 97%, or 98-99% identical to a nucleic acid of SEQ ID NO:8, 10, 12, or 14.

In certain embodiments, the methods and compositions disclosed herein may employ, as an BD-inducing agent, such as a viral protein. In certain embodiments, the viral protein is an HIV protein such as for example gp120 or gp41. In certain embodiments, an HIV protein is a polypeptide comprising an amino acid sequence at least 80%, 85%, 90%, 95%, 97%, or 98-99% identical to an amino acid sequence of SEQ ID NO:19, 20, or 21. In certain embodiments, an HIV protein is a polypeptide obtained by expressing a nucleic acid that is at least 80%, 85%, 90%, 95%, 97%, or 98-99% identical to a nucleic acid of SEQ ID NO:19, 20, or 21 in a cell. In certain embodiments, an HIV protein is a polypeptide encoded by a nucleic acid that is at least 80%, 85%, 90%, 95%, 97%, or 98-99% identical to a nucleic acid of SEQ ID NO:19, 20, or 21.

In certain embodiments, an BD agent or BD-inducing agent is administered systemically, such as by administration into the bloodstream. In certain embodiments the BD agent or BD-inducing agent is administered locally, such as to a portion of the body selected from the group consisting of: the mouth, the nasopharyngeal tract, the anus, the vagina, the penis, the skin, and the eye. In certain embodiments, the agent is administered to a mucous membrane. In certain preferred embodiments, the BD agent or BD-inducing agent is administered in a form selected from the group consisting of: a mouthwash, a toothpaste, an aerosol, a rectal or vaginal suppository, a rectal or vaginal cream, a rectal or vaginal film, a skin lotion, a condom, an eye drop, and an eye ointment. Optionally the agent is administered in combination with an additional antiviral agent, such as a reverse transcriptase inhibitor or a protease inhibitor.

In certain embodiments, the virus targeted is a virus that associates with the CXCR4 receptor, such as an HIV of the X4-type.

The embodiments and practices of the present invention, other embodiments, and their features and characteristics, will be apparent from the description, figures and claims that follow, with all of the claims hereby being incorporated by this reference into this Summary.

### BRIEF DESCRIPTION OF THE DRAWINGS

FIG. 1 shows the effects of human beta-defensin-1, -2 and -3 on infection of ghost CD4/CCR5/CXCR5 cells X4-type green fluorescent protein reporter HIV.

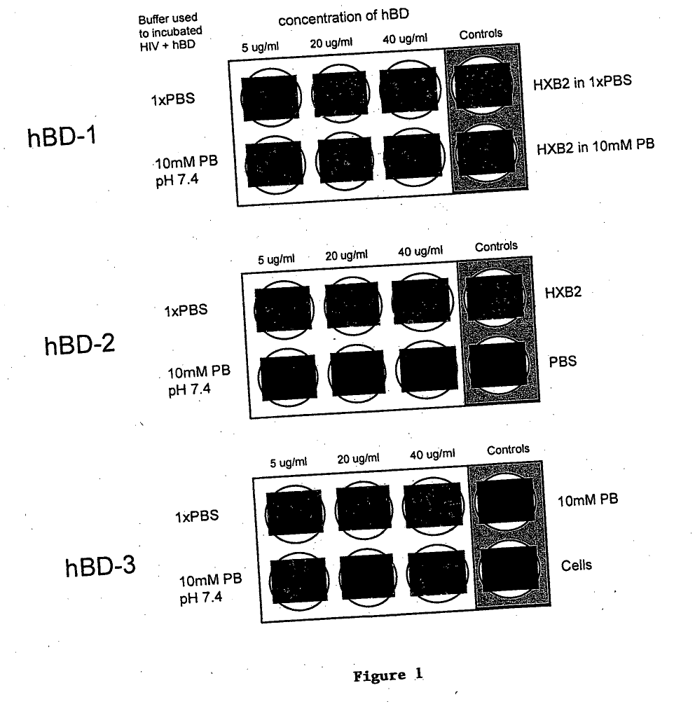

FIG. 2 shows the effect of HIV on the expression of HBD-2 and HBD-3 transcripts.

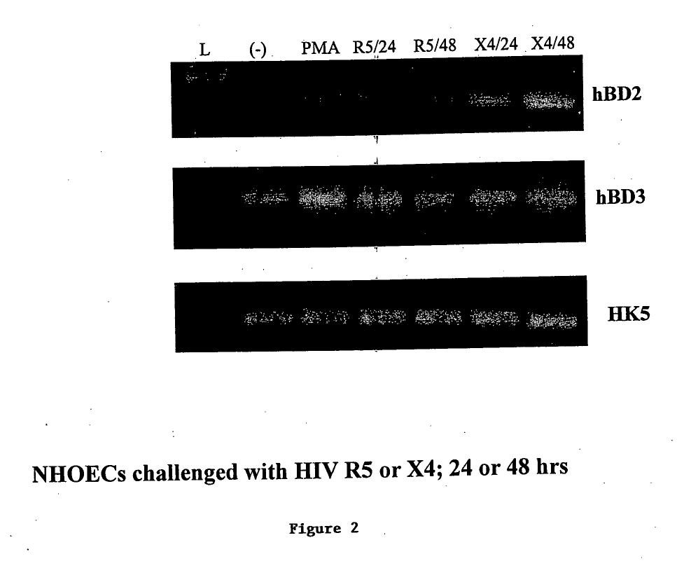

FIG. 3 shows HIV-1-induced expression of hBD-2 and -3. NHOEC monolayers were exposed to X4 HIV-1 strains B-HXB2 and B-NL4-3 or R5 strains B-93US142 and B-92US660 at an MOI of 0.01 infectious unit/cell. After 48 h, hBD-1, -2, and -3 mRNA expression was determined by real-time PCR. (a) Standard curves generated using the relationship of known number of input templates to the cycle threshold (i.e., PCR cycle number at which the mean fluorescence increases to 10 SD above baseline). Cycle threshold is directly proportional to the log of the input copy equivalents. Linear dynamic ranges and regression values are indicated. (b) Quantification of hBD mRNA in the presence and absence of HIV-1. (c) Comparison of hBD expression relative to the uninfected culture. PMA, phorbol myristate acetate, positive control. Results are representative of three independent experiments.

FIG. 4 shows anti-HIV-1 activity of hBD. HIV-1 strains (X4 HXB2 and R5 93US 142) were incubated in 10 mM PB with increasing concentrations of hBD and used to infect GHOST X4/R5 cells. (a) Qualitative determination of HIV-1 infection, measured by GFP fluorescence, in the absence (−C) and presence (+C) of virus preincubated in 10 mM PB. (b) Anti-HIV-1 activity of hBD in GHOST X4/R5 cells using fluorescence microscopy. (c) Anti-HIV-1 activity of hBD measured by RT activity in cell-free culture supernatant, relative to the positive control (i.e., HIV-1 infection in the absence of hBD). (d) Antiretroviral activity of hBD against CXCR4— and CCR5-tropic HIV-1 strains in three different environments: DMEM+10% FBS (complete medium); DMEM alone (Medium no FBS); 10 mM PB. Viruses were incubated with 20 μg/ml of each hBD for 1 h in each condition and used to infect CEM X4/R5 cells as described. Results are representative of three independent experiments.

FIG. 5 shows that hBD-2 and -3 downmodulate CXCR4. Unstimulated PBMC were treated for 3 hrs with hBD-1, -2, or -3 (30 μg/ml) in DMEM (high salt) in the absence of FBS. The CXCR4 natural ligand SDF-1 [symbol] (2 μg/ml) and the CCR5 antagonist PSC-RANTES (100 nM) were used as positive controls. CXCR4 and CCR5 surface expression was calculated using known ratios of QuantiBRITE-PE beads (Becton Dickenson) by flow cytometry. Results are the means of seven experiments±SD.

FIG. 6 shows that hBD-2 and -3 interact directly with HIV-1 and inhibit HIV-1 infectivity irreversibly. (a) CXCR4 tropic HXB2 and CCR5 tropic 97ZA003 HIV-1 strains were incubated with 20 μg/ml hBD-1, -2, or -3 in 10 mM PB for 1 h. Virions were pelleted, washed extensively with PBS and used to infect GHOST X4/R5 cells. RT activity was measured 48 h post-infection. The final two columns represent an additional 20 μg/ml of hBD-2 or -3 during infection. (b) Immunoelectron microscopy analysis showing the interaction of hBD-2 and -3 with HIV-1 and with MT4 cell membrane. X4 HIV-1HXB2 strain and MT4 cells were incubated with bBD-2 or -3 (20 μg/ml), 37° C., 1 h. Polyclonal anti-hBD-2 or -3 antibodies were added, followed by addition of secondary IgG conjugated with 10-nm gold particles. Arrows indicate hBD-2 and -3 localization to virions and cell membrane.

### DETAILED DESCRIPTION

1. Overview

In certain aspects, the present invention relates to the discovery that beta-defensin-2 (“BD-2”), beta-defensin-3 (“BD-3”), and related polypeptides, referred to herein as BD agents, inhibit the infection of cells by HIV. The invention also provides methods and compositions for inhibiting the ability of the HIV virus by regulating beta-defensin production. Beta-defensins are part of a family of small, cationic peptides that have anti-pathogenic effects against a broad range of pathogens, including bacteria, fungi, and viruses. Beta-defensins also have an effect on cancerous cells. Accordingly, agents that increase beta-defensin production can be used as preventative or therapeutic agents for a wide range of disorders.

Beta defensins are a superfamily of peptide antibiotics with a characteristic beta-sheet structure stabilized by two to three intramolecular disulfide bonds. They are strongly cationic by virtue of their numerous arginine and lysine residues. The human defensin family is divided into two subfamilies; alpha-defensins, found in azurophilic granules of PMNs and in the granules of Paneth cells found in the base of the crypts of Lieberkühn in the small intestine, and the beta-defensins, expressed primarily by epithelial cells. The beta-defensin subfamily was first described in columnar cells of bovine tracheal pseudostratified epithelium, and is now known to be expressed in various mucosal epithelia and organs. The alpha- and beta-defensins, differ in primary sequence and in the placement of the three disulfide bonds. The signature motif for beta-defensin genes includes two exons surrounding a variably sized intron. Exon 1 encodes the signal sequence, while exon 2 encodes the propeptide and mature peptide. This motif differs from that found in alpha-defensin genes in that the latter are organized with three exons and two introns. Other differentiating features between alpha- and beta-defensins include the fact that while the former are cytotoxic to mammalian cells when released from protective granules, the latter are not.

2. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

“Small molecule” as used herein, is meant to refer to a compound that has a molecular weight of less than about 5 kD and most preferably less than about 2.5 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures comprising arrays of small molecules, often fungal, bacterial, or algal extracts, which can be screened with any of the assays of the invention.

The term “compound” used herein is meant to include, but not limited to, peptides, nucleic acids, carbohydrates, small organic molecules, natural product extract libraries, and any other molecules (including, but not limited to, chemicals, metals and organometallic compounds).

A “chimeric polypeptide” or “fusion polypeptide” is a fusion of a first amino acid sequence with a second amino acid sequence where the first and second amino acid sequences are not naturally present in a single polypeptide chain.

An “expression construct” is any recombinant nucleic acid that includes an expressible nucleic acid and regulatory elements sufficient to mediate expression in a suitable host cell. For example, an expression construct may contain a promoter or other RNA polymerase contact site, a transcription start site or a transcription termination sequence. An expression construct for production of a protein may contain a translation start site, such as an ATG codon, a ribosome binding site, such as a Shine-Dalgarno sequence, or a translation stop codon.

The term “heterologous” as used in describing a nucleic acid with respect to another nucleic acid means that the two nucleic acids are not normally operably linked to each other or do not naturally occur in adjacent positions.

The term “including” is used herein to mean, and is used interchangeably with, the phrase “including but not limited to”.

The term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The term “or” is used herein to mean, and is used interchangeably with, the term “and/or”, unless context clearly indicates otherwise.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. Various alignment algorithms and/or programs may be used, including FASTA, BLAST or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

The terms “polypeptide” and “protein” are used interchangeably herein.

The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “substantially free of other contaminating proteins”) is defined as encompassing individual preparations of each of the component proteins comprising less than 20% (by dry weight) contaminating protein, and preferably comprises less than 5% contaminating protein. Functional forms of each of the component proteins can be prepared as purified preparations by using a cloned gene as described in the attached examples. By “purified”, it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 85% by weight, more preferably 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above.

The term “recombinant nucleic acid construct” includes any nucleic acid comprising at least two sequences which are not present together in nature. A recombinant nucleic acid may be generated in vitro, for example by using the methods of molecular biology, or in vivo, for example by insertion of a nucleic acid at a novel chromosomal location by homologous or non-homologous recombination.

3. Beta-Defensin Agents

The invention provides methods and compositions that employ BD agents. Certain embodiments employ HBD agents.

In certain embodiments, the methods and compositions disclosed herein may employ, as an HBD-2 agent, a polypeptide comprising an amino acid sequence at least 80%, 85%, 90%, 95%, 97%, or 98-99% identical to an amino acid sequence selected from the group consisting of: SEQ ID NO:1 and SEQ ID NO:2. In certain embodiments, the HBD-2 agent is a polypeptide obtained by expressing a nucleic acid that is at least 80%, 85%, 90%, 95%, 97%, or 98-99% identical to a nucleic acid selected from the group consisting of: SEQ ID NOs: 4-7 in a cell. In certain embodiments, the HBD-2 agent is a polypeptide encoded by a nucleic acid that is at least 80%, 85%, 90%, 95%, 97%, or 98-99% identical to a nucleic acid selected from the group consisting of: SEQ ID NOs:4-7. In certain embodiments, the HBD agent is a polypeptide of SEQ ID NO:1-2 or is encoded by a nucleic acid as set forth in SEQ ID Nos: 4-7. Preferably the HBD-2 agent has a 50% effectiveness at a concentration of about 10 micromolar or less.

In certain embodiments, the methods and compositions disclosed herein may employ, as an HBD-3 agent, a polypeptide comprising an amino acid sequence at least 80%, 85%, 90%, 95%, 97%, or 98-99% identical to an amino acid sequence selected from the group consisting of: SEQ ID NO:15. In certain embodiments, the HBD-3 agent is a polypeptide obtained by expressing a nucleic acid that is at least 80%, 85%, 90%, 95%, 97%, or 98-99% identical to a nucleic acid selected from the group consisting of: SEQ ID NOs:16-18 in a cell. In certain embodiments, the HBD-3 agent is a polypeptide encoded by a nucleic acid that is at least 80%, 85%, 90%, 95%, 97%, or 98-99% identical to a nucleic acid selected from the group consisting of: SEQ ID NOs:16-18. In certain embodiments, the HBD agent is a polypeptide of SEQ ID NO:15 or is encoded by a nucleic acid as set forth in SEQ ID Nos:16-18. Preferably the HBD-3 agent has a 50% effectiveness at a concentration of about 10 micromolar or less.

Exemplary HBD-2 Agents:

| | |
|---|---|
| GIGDPVTCLKSGAICHPVFCPRRYKQIGTCGLPGTKC | (SEQ ID NO:1) |
| CKKP | |
| MRVLYLLFSFLFIFLMPLPGVFGGIGDPVTCLKSGAI | (SEQ ID NO:2) |
| CHPVFCPRRYKQIGTCGLPGTKCCKKP | |

Exemplary HBD-3 Agent:

| | |
|---|---|
| MRIHYLLFALLFLFLVPVPGHGGIINTLQKYYCRVR | (SEQ ID NO:15) |
| GGRCAVLSCLPKEEQIGKCSTRGRKCCRRKK | |

The subject BD agents also encompass nucleic acids that encode BD polypeptides or portions thereof.

In certain aspects the invention involves the use of isolated and/or recombinant nucleic acids encoding BD polypeptides, such as, for example, SEQ ID NOs: 4-7 and 16-18. Certain methods of the invention are further understood to employ nucleic acids that comprise variants of SEQ ID NOs: 4-7 and 16-18. Variant nucleotide sequences include sequences that differ by one or more nucleotide substitutions, additions or deletions, such as allelic variants; and will, therefore, include coding sequences that differ from the nucleotide sequence of the coding sequence designated in SEQ ID NOs: 4-7 and 16-18, e.g. due to the degeneracy of the genetic code. For example, nucleic acids encoding HBD-2 polypeptides may be nucleic acids comprising a sequence that is at least 90%, 95%, 99% or 10% identical to the sequence of SEQ ID NOs:4-7 or a sequence that encodes the polypeptide of SEQ ID NOs:1 or 2. In other embodiments, variants will also include sequences that will hybridize under highly stringent conditions to a coding sequence of a nucleic acid sequence designated in SEQ ID NOs: 4-7.

Exemplary HBD-2 Gene Sequence (SEQ ID NO:4)

| | |
|---|---|
| 1 | ctttataagg tggaaggctt gatgtcctcc ccagactcag ctcctggtga agctcccagc |
| 61 | catcagccat gagggtcttg tatctcctct tctcgttcct cttcatattc ctgatgcctc |
| 121 | ttccaggtga gatgggccag ggaaatagga gggttggcca aatggaagaa tggcgtagaa |
| 181 | gttctctgtc tcctctcatt cccctccacc tatctctccc tcatccctct ctctccttcc |
| 241 | tctctctgtg tgtcccctcc atccttttct cctgcttctc tctcttcttc cctctctctc |
| 301 | ttttttctgt ctttcttttt cctctctccc tagagcatgt ctttctttct ttctctttcc |
| 361 | tttcttctac ccacactttt agactgaatg ccctatttaa ttgaacaaag cattgcttcc |
| 421 | ttcaatagaa aaggagtttg agaacccaat ggacacctca ctcgttcttc taagccaata |
| 481 | tgaaggagcc cagtagcttg taaatatcat ctcttcactg ctttccatgc tacaactgct |
| 541 | gagactatgg ttgaaacctg ttaggtgact ttttaaataa aaggcagaaa ttttgatttt |
| 601 | atctaaagaa agtagtatag aatgtcattt tctaaatttt tatatttaaa gggtagatac |
| 661 | tgcaacctag agaattccag ataatcttaa ggcccagcct atactgtgag aactactgca |
| 721 | gcaagacact ctgcctccag gacttttctg atcagaggcc ctgagaacag tccctgccac |
| 781 | taggccactg caggttcaca ggacagggta cagcccattg aaacctactt ttaaacctgg |
| 841 | atgcctaacc ttcattttct ccttgatatt atgaaaataa aataaaaacc atgaaaggat |
| 901 | aaaagaggga gagtggaagg gaaggatgga gaaagggaaa aagaaaattt gagagtaaat |
| 961 | cctaaaacaa ttaatctaat agatatcatc ttgtgaaatc ctcattttac caatcttatt |
| 1021 | tatgagtcct gggttttgtg agaacaatgg ggttctgaga ggcaccagag acctcatgtt |
| 1081 | ttccaaaacc tagaacagta taatgaagga aggcggggag gcagggaggc agggaggcag |
| 1141 | ggaggcaggg aggcgggcag gtggggaggg agggacggaa ggagggaggg agggagggag |
| 1201 | ggagggaggg agggataaaa aaagaagaat gaggttgaaa ccaggactta gatattagaa |
| 1261 | acaagccatt acaaaattta tttctatggt taattgtggt tttcaactgt aagttacttg |
| 1321 | gtgttaattt cctattaaac aatttcagta agttgcatct ttttatccca tctcaggtca |
| 1381 | aatacttaac agactaaatg atttgaaaaa gcaaaagttt actggcttgt gtgtgttaaa |
| 1441 | atggaggtat ggtggctttg atattatctt cttgtggtgg agctgaattc acaagagatc |
| 1501 | gttgctgagc tcctaccaga ccccacctgg aggccccagt cactcaggag agatcagggt |
| 1561 | ctttcacaat caggttctac aaaaataaac atccccccaa ccacagcagt gccagtttcc |
| 1621 | atgtcagaaa cttagatcca aatgactgac tcgcgtctca ttatcatgat ggaaaagccc |
| 1681 | aggcttgaga aagaagcccg ctgcggattt actcaaggcg atactgacac agggtttgtg |
| 1741 | tttttccaac atgagttttg agttcttaca cgctgtttgc tctttttgtg tgttttttcc |
| 1801 | ctgttaggtg tttttggtgg tataggcgat cctgttacct gccttaagag tggagccata |
| 1861 | tgtcatccag tcttttgccc tagaaggtat aaacaaattg gcacctgtgg tctccctgga |
| 1921 | acaaaatgct gcaaaaagcc atgaggaggc caagaagctg ctgtggctga tgcggattca |
| 1981 | gaaagggctc cctcatcaga gacgtgcgac atgtaaacca aattaaacta tggtgtccaa |
| 2041 | agata |

Exemplary HBD-2 mRNA (SEQ ID NO:5)

| | |
|---|---|
| 1 | ggtgaagctc ccagccatca gccatgaggg tcttgtatct cctcttctcg ttcctcttca |
| 61 | tattcctgat gcctcttcca ggtgtttttg gtggtatagg cgatcctgtt acctgcctta |
| 121 | agagtggagc catatgtcat ccagtctttt gccctagaag gtataaacaa attggcacct |
| 181 | gtggtctccc tggaacaaaa tgctgcaaaa agccatgagg aggccaagaa gctgctgtgg |
| 241 | ctgatgcgga ttcagaaagg gctccctcat cagagacgtg cgacatgtaa accaaattaa |
| 301 | actatggtgt ccaaagata |

Exemplary HBD-2 Coding Sequence (Precursor) (SEQ ID NO:6)

| | |
|---|---|
| 1 | atgagggtct tgtatctcct cttctcgttc ctcttcatat tcctgatgcc tcttccaggt |
| 61 | gtttttggtg gtataggcga tcctgttacc tgccttaaga gtggagccat atgtcatcca |
| 121 | gtcttttgcc ctagaaggta taaacaaatt ggcacctgtg gtctccctgg aacaaaatgc |
| 181 | tgcaaaaagc catga |

Exemplary HBD-2 Coding Sequence (Mature) (SEQ ID NO:7)

| | |
|---|---|
| 1 | ggtataggcg atcctgttac ctgccttaag agtggagcca tatgtcatcc agtcttttgc |
| 61 | cctagaaggt ataaacaaat tggcacctgt ggtctccctg gaacaaaatg ctgcaaaaag |
| 121 | ccatga |

Exemplary HBD-3 Coding Sequence (SEQ ID NO: 16)

| | |
|---|---|
| 1 | atgaggatcc attatcttct gtttgctttg ctcttcctgt ttttggtgcc tgttccaggt |
| 61 | catggaggaa tcataaacac attacagaaa tattattgca gagtcagagg cggccggtgt |
| 121 | gctgtgctca gctgccttcc aaaggaggaa cagatcggca agtgctcgac gcgtggccga |
| 181 | aaatgctgcc gaagaaagaa ataa |

Exemplary HBD-3 Gene Sequence (SEQ ID NO:17)

| | |
|---|---|
| 1 | tgagtctcag cgtggggtga agcctagcag ctatgaggat ccattatctt ctgtttgctt |
| 61 | tgctcttcct gtttttggtg cctgtcccag gtcatggagg aatcataaac acattacaga |
| 121 | aatattattg cagagtcaga ggcggccggt gtgctgtgct cagctgcctt ccaaaggagg |
| 181 | aacagatcgg caagtgctcg acgcgtggcc gaaaatgctg ccgaagaaag aaataaaaac |
| 241 | cctgaaacat gacgagagtg ttgtaaagtg tggaaatgcc ttcttaaagt ttataaaagt |
| 301 | aaaatcaaat tacatttttt tttcaaaaaa aaaaaaa |

Exemplary HBD-3 mRNA Sequence (SEQ ID NO:18)

| | |
|---|---|
| 1 | catccagtct cagcgtgggg tgaagcctag cagctatgag gatccattat cttctgtttg |
| 61 | ctttgctctt cctgtttttg gtgcctgttc caggtcatgg aggaatcata aacacattac |
| 121 | agaaatatta ttgcagagtc agaggcggcc ggtgtgctgt gctcagctgc cttccaaagg |
| 181 | aggaacagat cggcaagtgc tcgacgcgtg gccgaaaatg ctgccgaaga aagaaataaa |
| 241 | aaccctgaaa catgacgaga gtgttg |

Recently, a computational search strategy identified 28 new human β-defensin genes in five syntenic chromosomal regions. Schutte, B. C., J. P. Mitros, J. A. Bartlett, J. D. Walters, H. P. Jia, M. J. Welsh, T. L. Casavant, and P. B. McCray, Jr. Discovery of five conserved beta-defensin gene clusters using a computational search strategy. 2002. Proc Natl Acad Sci USA 99:2129. At least 26 of the predicted genes were found to be transcribed. This study focused on finding β-defensin second exons, the genetic region encoding the mature peptide. It is anticipated that a similar approach could be used to discover all first exon coding sequences and the associated regulatory elements that confer c ell specificity and responsiveness to inflammatory stimuli and pathogens. These new findings provide additional candidate BD like agents.

4. Beta-Defensin Inducing Agents

The invention also provides BD-inducing agents and screening assays to identify candidate agents that may be BD-inducing agents. A BD-inducing agent is an agent that induces a beta defensin such as for example BD-2 or BD-3. In certain embodiments, the BD-inducing agent induces a HBD-2 or HBD-3. A BD-inducing agent may be selected from the group consisting of a polypeptide and portions thereof, a fusion protein, a small molecule, a peptidomimetic, and/or a nucleic acid agent.

In yet other embodiments, the BD-inducing agent induces a polypeptide comprising an amino acid sequence at least 80%, 85%, 90%, 95%, 97%, or 98-99% identical to an amino acid sequence selected from the group consisting of: SEQ ID NO:1 and SEQ ID NO:2. In certain embodiments, the BD-inducing agent induces a polypeptide obtained by expressing a nucleic acid that is at least 80%, 85%, 90%, 95%, 97%, or 98-99% identical to a nucleic acid selected from the group consisting of: SEQ ID NOs: 4-7 in a cell. In certain embodiments, the BD-inducing agent induces a polypeptide encoded by a nucleic acid that is at least 80%, 85%, 90%, 95%, 97%, or 98-99% identical to a nucleic acid selected from the group consisting of: SEQ ID NOs:4-7.

In yet other embodiments, a BD-inducing agent induces a polypeptide comprising an amino acid sequence at least 80%, 85%, 90%, 95%, 97%, or 98-99% identical to an amino acid sequence of: SEQ ID NO:15. In certain embodiments, a BD-inducing agent induces a polypeptide obtained by expressing a nucleic acid that is at least 80%, 85%, 90%, 95%, 97%, or 98-99% identical to a nucleic acid selected from the group consisting of: SEQ ID NOs:16-18 in a cell. In certain embodiments, a BD-inducing agent induces a polypeptide encoded by a nucleic acid that is at least 80%, 85%, 90%, 95%, 97%, or 98-99% identical to a nucleic acid selected from the group consisting of: SEQ ID NOs:16-18.

In certain embodiments, methods and compositions disclosed herein may employ, as an BD-inducing agent, a Fusobacterium Associated Defensin Inducer polypeptide (FAD-I). In certain embodiments, an FAD-I polypeptide is a polypeptide comprising an amino acid sequence at least 90%, 95%, 97%, 99% or 100% identical to an amino acid sequence of SEQ ID NO: 3, 9, 11, or 13, wherein said polypeptide is sufficient to induce beta-defensin 2 or 3 (BD-2 or -3) production, and preferably induction of human beta-defensin 2 or 3 (hBD-2 or -3) production. In certain embodiments, an FAD-I polypeptide is a polypeptide obtained by expressing a nucleic acid that is at least 90%, 95%, 97%, 99% or 100% identical to a nucleic acid of SEQ ID NO:8, 10, 12, or 14 in a cell, preferably a bacterial cell, such as F. nucleatum or E. coli. In certain embodiments, a FAD-I polypeptide is a polypeptide encoded by a nucleic acid that is at least 90%, 95%, 97%, 99% or 100% identical to a nucleic acid of SEQ ID NO:8, 10, 12, or 14. In certain embodiments, a FAD-I polypeptide is a polypeptide derived from a F. nucleatum cell wall, having a monomeric molecular weight range of about 12-14 kDa and which polypeptide induces BD-2 or BD-3 production. In certain embodiments, the FAD-I polypeptide additionally has a pI of between 4.0 and 5.5. In certain embodiments a FAD-I polypeptide is purified or partially purified. In preferred embodiments, the FAD-I polypeptide and/or a composition comprising the FAD-I polypeptide induces beta-defensin production in at least one epithelial cell type, such as an oral epithelial cell, a corneal epithelial cell, a skin cell. In preferred embodiments, the defensin induced is a BD-2 or BD-3, and in humans, an HBD-2 or HBD-3. In certain embodiments, the FAD-I polypeptide and/or composition comprising the FAD-I polypeptide induces beta-defensin production in one or more cells of a mucosal epithelium, such as the vagina, rectum, urethra, intestines, nasal epithelium, oral epithelium or corneal epithelium.

In certain embodiments, the methods and compositions disclosed herein may employ, as an BD-inducing agent, such as a viral protein. In certain embodiments, the viral protein is an HIV protein such as for example gp120 or gp41. In certain embodiments, an HIV protein is a polypeptide comprising an amino acid sequence at least 80%, 85%, 90%, 95%, 97%, or 98-99% identical to an amino acid sequence of SEQ ID NO:19, 20, or 21. In certain embodiments, an HIV protein is a polypeptide obtained by expressing a nucleic acid that is at least 80%, 85%, 90%, 95%, 97%, or 98-99% identical to a nucleic acid of SEQ ID NO:19, 20, or 21 in a cell. In certain embodiments, an HIV protein is a polypeptide encoded by a nucleic acid that is at least 80%, 85%, 90%, 95%, 97%, or 98-99% identical to a nucleic acid of SEQ ID NO:19, 20, or 21.

Exemplary HBD-Inducing Agents:

SEQ ID NO.:3 (NP_602592)

MSLFLVACGEKKEEEKPAEQAAVEATATEAPATETTEAAAEAKTFSLKTE
DGKEFTLVVAADGSTATLTDAEGKATELKNAETASGERYADEAGNEVAMK
GAEGILTLGDLKEVPVTVEAK

SEQ ID NO.:9 (NP_602354)

MKKILLLLSSLFLFACANIDTGVDESKEAQISRLLKEADKKKEKTVEVEK
KLVTDNGEEVIEEEATVQNKKSHKGMTRGEIMEYEMTRVSDEMNALQADV
QQYQEKKAQLKAYQEKLQKLEELNNAGIK

SEQ ID NO.:11 (NP_602356)

MKKVILTLFVLLSIGIFANDEIISELKGLNAEYENLVKEEEARFQKEKEL
SERAAAQNVKLAELKASIEEKLLAAPEERKTKFFKDTFDGLVKDYSKYLS
QINEKIAENTEIVSNFEKIQKIR

SEQ ID NO.:13 (NP_603171)

MKKFLLLAVLAVSASAFAANDAASLVGELQALDAEYQNLANQEEARFNEE
RAQADAARQALAQNEQVYNELSQRAQRLQAEANTRFYKSQYQDLASKYED
ALKKLESEMEQQKAIISDFEKIQALRAGN
Exemplary gp120/gp41:

SEQ ID NO:19

SLWDQSLKPCVKLTPLCVTLNCRDVNATNTGNVTYNDTIKGEIKNCSFNT
TTEIRDKKQTAYALFYKLDIVPLNDGNNNNXY

SEQ ID NO:20

SLWDQSLKPCVKLTPLCVTLXCXNATFNNITTFNIXNSSSNITTYPINNT
TNQHSLFYNLHVLP

SEQ ID NO:21

SLWDQSLKPCVKLTPLCVTLKCENATINNGGNATVASNDTINREVKNCSF
NITTDLRDKRKHEYALFYTLDIVPLNEKKNNASEYRLISCNTSAVTQACP
K

In certain aspects the invention involves the use of isolated and/or recombinant nucleic acids encoding a BD-inducing polypeptide or protein such as FAD-I polypeptides or gp120 o gp41 polypeptides. Exemplary nucleic acids encoding FAD-I polypeptides are set forth in SEQ ID NOs: 8, 10, 12, or 14. Certain methods of the invention are further understood to employ nucleic acids that comprise variants of SEQ ID NOs: 8, 10, 12, or 14. Variant nucleotide sequences include sequences that differ by one or more nucleotide substitutions, additions or deletions, such as allelic variants; and will, therefore, include coding sequences that differ from the nucleotide sequence of the coding sequence designated in SEQ ID NOs:8, 10, 12, or 14, e.g. due to the degeneracy of the genetic code. For example, nucleic acids encoding FAD-I polypeptides may be nucleic acids comprising a sequence that is at least 90%, 95%, 99% or 10% identical to the sequence of SEQ ID NOs:8, 10, 12, or 14, or a sequence that encodes the polypeptide of SEQ ID NOs:3, 9, 11, or 13. In other embodiments, variants will also include sequences that will hybridize under highly stringent conditions to a coding sequence of a nucleic acid sequence designated in SEQ ID NOs: 8, 10, 12, or 14.

Exemplary HBD-Inducing Nucleic Acids:

SEQ ID NO:8 (Nucleotides 272989-273354 of NC_{—}003454):

atgagtttattcttagtagcttgtggagaaaaaaaagaagaagaaaaacc
agctgaacaagctgctgtagaagcaactgcaactgaagcacctgctacag
aaacaactgaagctgctgctgaagctaaaacattctcacttaaaactgaa
gatggaaaagaattcacattagtagttgctgctgatggaagtactgcaac
tttaactgatgcagaaggaaaagcaactgaattaaaaaatgctgaaactg
catctggagaaagatatgcagatgaagctggaaacgaagttgctatgaaa
ggtgcagaaggaatcttaactttaggagaccttaaagaagtaccagtaac
tgttgaagctaaatag

SEQ ID NO:10 (Nucleotides 42273-2662 of NC_{—}003454):

ttgaaaaaaatattattactattatcttctttatttttatttgcttgtgc
taatatagatacaggtgtagatgaaagtaaagaagctcaaatatcaagac
ttttaaaagaagctgataagaaaaaagaaaaaacagtagaagtagaaaag
aaacttgtaactgataatggagaggaagttatagaggaagaagctaccgt
tcaaaacaaaaaatcacataaaggaatgacaagaggggaaataatggaat
atgaaatgacaagagtttcagatgaaatgaatgccctacaagcggatgta
caacaatatcaagaaaagaaagcacaactaaaagcataccaagaaaaatt
acaaaaattagaagaattaaataatgcaggaataaaataa

SEQ ID NO:12 (Nucleotides 43083-43454 of NC_{—}003454):

atgaaaaaagttattttaacattatttgttttattatctattggaatatt
tgcaaatgatgagattatttcagagttaaaaggacttaatgctgagtatg
aaaatttagtaaaagaagaagaagctagatttcaaaaagaaaaagaactt
tctgaaagagcagcagctcaaaatgttaaattggctgaattaaaagcaag
cattgaagaaaaattgttagcagctccagaagaaagaaaaacaaaatttt
ttaaagatacttttgatggtttagtgaaagattattcaaaatatttaagt
caaataaatgaaaaaatagctgaaaatactgaaatagtaagtaattttga
aaaaattcaaaaaataagatag

SEQ ID NO:14 (Nucleotides 891002-891391 of NC_{—}003454):

atgaaaaaatttttattattagcagtattagctgtttctgcttcagcatt
cgcagcaaatgatgcagcaagtttagtaggtgaattacaagcattagatg
ctgaataccaaaacttagcaaatcaagaagaagcaagattcaatgaagaa
agagcacaagctgacgctgctagacaagcactagcacaaaatgaacaagt
ttacaatgaattatctcaaagagctcaaagacttcaagctgaagctaaca
caagattttataaatctcaataccaagatctagcttctaaatatgaagac
gctttaaagaaattagaatctgaaatggaacaacaaaaagctattatttc
tgattttgaaaaaattcaagctttaagagctggtaactaa

One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the invention provides nucleic acids which hybridize under low stringency conditions of 6×SSC at room temperature followed by a wash at 2×SSC at room temperature.

Isolated nucleic acids which differ from those described above due to degeneracy in the genetic code are also within the scope of the invention. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among Fusobacterium cultivars. One skilled in the art will appreciate that these variations in one or more nucleotides of the nucleic acids encoding a particular protein may exist among individuals of a given species due to natural allelic variation. Any and all such nucleotide variations and resulting amino acid polymorphisms are within the scope of this invention.

Optionally, an BD-inducing nucleic acid of the invention will genetically complement a partial or complete loss of function phenotype in an F. nucleatum cell. For example, a FAD-I nucleic acid of the invention may be expressed in a cell in which endogenous FAD-I has been knocked out, and the introduced FAD-I nucleic acid will mitigate a phenotype resulting from the knockout. An exemplary FAD-I loss of function phenotype is a decrease in the stimulation of BD-2 or BD-3 expression in cells (e.g., NHOECs) or similarly sensitive cell types.

In certain aspects, nucleic acids encoding BD or BD-inducing polypeptides may be used to increase BD or BD-inducing gene expression in an organism or cell by direct delivery of the nucleic acid. A nucleic acid therapy construct of the present invention can be delivered, for example, as an expression plasmid which, when transcribed in the cell, produces RNA which encodes a BD or BD-inducing polypeptide.

In another aspect of the invention, the subject nucleic acid is provided in an expression vector comprising a nucleotide sequence encoding a subject BD or BD-inducing polypeptide and operably linked to at least one regulatory sequence. Regulatory sequences are art-recognized and are selected to direct expression of the BD or BD-inducing polypeptide. Accordingly, the term regulatory sequence includes promoters, enhancers and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding a HBD or HBD-inducing polypeptide. Such useful expression control sequences, include, for example, the early and late promoters of SV40, tet promoter, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

As will be apparent, the subject gene constructs can be used to cause expression of the subject BD or BD-inducing polypeptides in cells propagated in culture, e.g. to produce proteins or polypeptides, including fusion proteins or polypeptides, for purification.

This invention also pertains to a host cell transfected with a recombinant gene including a coding sequence for one or more of the subject BD or BD-inducing polypeptides. The host cell may be any prokaryotic or eukaryotic cell. For example, a polypeptide of the present invention may be expressed in bacterial cells such as E. coli, insect cells (e.g., using a baculovirus expression system), yeast, or mammalian cells. Other suitable host cells are known to those skilled in the art.

Accordingly, the present invention further pertains to methods of producing the subject BD or BD-inducing polypeptides. For example, a host cell transfected with an expression vector encoding a HBD or HBD-inducing polypeptide can be cultured under appropriate conditions to allow expression of the polypeptide to occur. The polypeptide may be secreted and isolated from a mixture of cells and medium containing the polypeptide. Alternatively, the polypeptide may be retained cytoplasmically and the cells harvested, lysed and the protein isolated. A cell culture includes host cells, media and other byproducts. Suitable media for cell culture are well known in the art. The polypeptide can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptide. In a preferred embodiment, the BD or BD-inducing polypeptide is a fusion protein containing a domain which facilitates its purification, such as a BD or BD-inducing-GST fusion protein, HBD or HBD-inducing-intein fusion protein, BD or BD-inducing-cellulose binding domain fusion protein, HBD or HBD-inducing-polyhistidine fusion protein etc.

Methods for purifying FAD-I from F. nucleatum cell wall extracts are also disclosed herein. The methods may involve growing anaerobic cultures of F. nucleatum ATCC 25586, preparing their crude cell wall extract using a French pressure cell at 15,000 lb/in2 and differential centrifugation as previously described [Krisanaprakornkit et al. 1998], a nd further purification with an HPLC system. For example, the crude extract may be applied to a C4 HPLC column and fractions may be eluted with an acetonitrile gradient. The fractions may be further tested for the presence of an BD-inducing agent, e.g., a FAD-I.

In certain embodiments, small molecules are candidate agents to be screened for identifying BD-inducing agents. In certain preferred embodiments, small molecules are generated by combinatorial synthesis.

The candidate agents used in the invention may be pharmacologic agents already known in the art or may be agents previously unknown to have any pharmacological activity. The agents may be naturally arising or designed or prepared in the laboratory. They may be isolated from microorganisms, animals, or plants, or may be produced recombinantly, or synthesized by chemical methods known in the art. In some embodiments, candidate agents are identified from small chemical libraries, peptide libraries, or collections of natural products using the methods of the present invention. Tan et al. described a library with over two million synthetic compounds that is compatible with miniaturized cell-based assays (J. Am. Chem. Soc. 120, 8565-8566, 1998). It is within the scope of the present invention that such a library may be used to screen for agents that are HBD-inducing agents using the methods of the invention. There are numerous commercially available compound libraries, such as the Chembridge DIVERSet. Libraries are also available from academic investigators, such as the Diversity set from the NCI developmental therapeutics program.

One basic approach to search for a subject agent is screening of compound libraries. One may simply acquire, from various commercial sources, small molecule libraries that are believed to meet the basic criteria for useful drugs in an effort to identify useful compounds by “brute force.” Screening of such libraries, including combinatorially generated libraries, is a rapid and efficient way to screen a large number of related (and unrelated) compounds for activity. Combinatorial approaches also lend themselves to rapid evolution of potential drugs by the creation of second, third, and fourth generation compounds modeled on active but otherwise undesirable compounds. It will be understood that undesirable compounds include compounds that are typically toxic, but have been modified to reduce the toxicity or compounds that typically have little effect with minimal toxicity and are used in combination with another compound to produce the desired effect.

Another aspect of the disclosure relates to polypeptides derived from a full-length BD or BD-inducing polypeptide. Isolated peptidyl portions of the subject proteins can be obtained by screening polypeptides recombinantly produced from the corresponding fragment of the nucleic acid encoding such polypeptides. In addition, fragments can be chemically synthesized using techniques known in the art such as conventional Merrifield solid phase f-Moc or t-Boc chemistry. For example, any one of the subject proteins can be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function in a cellular assay for BD-2 induction or BD-3 induction or both.

It is also possible to modify the structure of the subject BD or BD-inducing polypeptides for such purposes as enhancing therapeutic or prophylactic efficacy, or stability (e.g., ex vivo shelf life and resistance to proteolytic degradation in vivo). Such modified polypeptides, when designed to retain at least one activity of the naturally-occurring form of the protein, are considered functional equivalents of the BD or BD-inducing polypeptides described in more detail herein. Such modified polypeptides can be produced, for instance, by amino acid substitution, deletion, or addition.

For instance, it is reasonable to expect, for example, that an isolated replacement of a leucine with an isoleucine or valine, an aspartate with a glutamate, a threonine with a serine, or a similar replacement of an amino acid with a structurally related amino acid (i.e. conservative mutations) will not have a major effect on the biological activity of the resulting molecule. Conservative replacements are those that take place within a family of amino acids that are related in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2nd ed., Ed. by L. Stryer, W.H. Freeman and Co., 1981). Whether a change in the amino acid sequence of a polypeptide results in a functional homolog can be readily determined by assessing the ability of the variant polypeptide to produce a response in cells in a fashion similar to the wild-type protein. For instance, such variant forms of BD-inducing polypeptide can be assessed, e.g., for their ability induce BD-2 production in a cell. Such variant forms of BD polypeptide can be assessed, e.g., for their ability to inhibit HIV infection of cells or to kill certain target bacteria such as Porphyromonas gingivalis. Polypeptides in which more than one replacement has taken place can readily be tested in the same manner.

This invention further contemplates a method of generating sets of combinatorial mutants of the subject BD or BD-inducing polypeptides, as well as truncation mutants. The purpose of screening such combinatorial libraries is to generate, for example, BD or BD-inducing homologs which can act as either agonists or antagonist, or alternatively, which possess novel activities all together. Combinatorially-derived homologs can be generated which have a selective potency relative to a naturally occurring BD or BD-inducing polypeptide. Such proteins, when expressed from recombinant DNA constructs, can be used in gene therapy protocols.

Likewise, mutagenesis can give rise to homologs which have intracellular half-lives dramatically different than the corresponding wild-type protein. For example, the altered protein can be rendered either more stable or less stable to proteolytic degradation or other cellular process which result in destruction of, or otherwise inactivation of the BD or BD-inducing polypeptide of interest.

In similar fashion, BD or BD-inducing homologs can be generated by the present combinatorial approach to act as antagonists, in that they are able to interfere with the ability of the corresponding wild-type protein to function.

In a representative embodiment of this method, the amino acid sequences for a population of BD or BD-inducing homologs are aligned, preferably to promote the highest homology possible. Such a population of variants can include, for example, homologs from one or more species of Fusobacterium, or homologs from the same species but which differ due to mutation. Amino acids which appear at each position of the aligned sequences are selected to create a degenerate set of combinatorial sequences. In a preferred embodiment, the combinatorial library is produced by way of a degenerate library of genes encoding a library of polypeptides which each include at least a portion of potential BD or BD-inducing sequences. For instance, a mixture of synthetic oligonucleotides can be enzymatically ligated into gene sequences such that the degenerate set of potential BD or BD-inducing nucleotide sequences are expressible as individual polypeptides, or alternatively, as a set of larger fusion proteins (e.g. for phage display).

There are many ways by which the library of potential homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential BD or BD-inducing sequences. The synthesis of degenerate oligonucleotides is well known in the art (see for example, Narang, S A (1983) Tetrahedron 39:3; Itakura et al., (1981) Recombinant DNA, Proc. 3rd Cleveland Sympos. Macromolecules, ed. AG Walton, Amsterdam: Elsevier pp273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, BD or BD-inducing variants can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated and bioactive variants of BD or BD-inducing polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of BD or BD-inducing variants. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

BD or BD-inducing polypeptides may further comprise post-translational or non-amino acid elements, such as hydrophobic modifications (e.g. polyethylene glycols or lipids), poly- or mono-saccharide modifications, phosphates, acetylations, etc. Effects of such elements on the functionality of a FAD-I polypeptide may be tested as described herein for other FAD-I variants.

The disclosure further provides methods for testing the functionality of BD-inducing polypeptides, such as FAD-I polypeptides or gp120/gp41 polypeptides, variants and fragments. In general, cells may be transfected with a BD-2 or BD-3 reporter construct, wherein a FAD-I responsive regulatory element of a BD-2 or BD-3 gene is operably linked to a reporter gene, and preferably a reporter gene that produces a fluorescent protein (e.g. green fluorescent protein) or an enzyme that can generate a fluorescent substrate. The cells are then contacted with the FAD-I polypeptide and reporter gene expression is assessed. In certain embodiments, an assay may comprise employing a cell that naturally has inducible BD-2 or BD-3 expression, such as a normal human oral epithelial cell. The cell may be transfected with a reporter construct or the expression of normal BD-2 or BD-3 transcript or polypeptide may be assessed.

The invention also provides for reduction of the subject BD agents or BD-inducing polypeptides or proteins to generate mimetics, e.g., peptide or non-peptide agents, which are able to mimic action of the authentic protein in a host. Such mutagenic techniques as described herein, as well as the thioredoxin system, are also particularly useful for mapping the determinants of a BD agent which participates in protein-protein interactions involved in, for example, binding of a BD to a CXCR4. To illustrate, the critical residues of a BD polypeptide or protein such as for example a BD-2 and/or BD-3 polypeptide or protein can be determined and used to generate its derived peptidomimetics which can affect the binding between the BD polypeptide or protein with another protein such as CXCR4. In other aspects, the critical residues of a BD-inducing polypeptide or protein can be determined with which can induce expression of a BD such as for example a BD-2 and/or BD-3. By employing, for example, scanning mutagenesis to map the amino acid residues of a BD which are involved in binding to another polypeptide such as CXCR4 or the residues of a BD-inducing polypeptide or protein which can induce expression of a BD, peptidomimetic compounds can be generated which mimic those residues involved in binding or inducing expression of a BD. Non-hydrolyzable peptide analogs of such residues can be generated using benzodiazepine (e.g., see Freidinger et al., in Peptides: Chemistry and Biology, G. R. Marshall ed., ESCOM Publisher: Leiden, Netherlands, 1988), azepine (e.g., see Huffman et al., in Peptides: Chemistry and Biology, G. R. Marshall ed., ESCOM Publisher: Leiden, Netherlands, 1988), substituted gamma lactam rings (Garvey et al., in Peptides: Chemistry and Biology, G. R. Marshall ed., ESCOM Publisher: Leiden, Netherlands, 1988), keto-methylene pseudopeptides (Ewenson et al., (1986) J. Med. Chem. 29:295; and Ewenson et al., in Peptides: Structure and Function (Proceedings of the 9th American Peptide Symposium) Pierce Chemical Co. Rockland, Ill., 1985), b-turn dipeptide cores (Nagai et al., (1985) Tetrahedron Lett 26:647; and Sato et al., (1986) J Chem Soc Perkin Trans 1:1231), and b-aminoalcohols (Gordon et al., (1985) Biochem Biophys Res Commun 126:419; and Dann et al., (1986) Biochem Biophys Res Commun 134:71).

A nucleotide sequence encoding an BD or BD-inducing polypeptide can be used to produce a recombinant form of the protein via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial) cells, are standard procedures.

A recombinant BD-2 or BD-inducing nucleic acid can be produced by ligating the cloned gene, or a portion thereof, into a vector suitable for expression in either prokaryotic cells, eukaryotic cells, or both. Expression vehicles for production of a recombinant HBD or HBD-inducing polypeptides include plasmids and other vectors. For instance, suitable vectors for the expression of a FAD-I polypeptide include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al., (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used.

The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant FAD-I polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

It is well known in the art that a methionine at the N-terminal position can be enzymatically cleaved by the use of the enzyme methionine aminopeptidase (MAP). MAP has been cloned from E. coli (Ben-Bassat et al., (1987) J. Bacteriol. 169:751-757) and Salmonella typhimurium and its in vitro activity has been demonstrated on recombinant proteins (Miller et al., (1987) PNAS USA 84:2718-1722). Therefore, removal of an N-terminal methionine, if desired, can be achieved either in vivo by expressing such recombinant polypeptides in a host which produces MAP (e.g., E. coli or CM89 or S. cerevisiae), or in vitro by use of purified MAP (e.g., procedure of Miller et al.).

In another embodiment, a fusion gene coding for a purification leader sequence, such as a poly-(His)/enterokinase cleavage site sequence at the N-terminus of the desired portion of the recombinant protein, can allow purification of the expressed fusion protein by affinity chromatography using a Ni²⁺ metal resin. The purification leader sequence can then be subsequently removed by treatment with enterokinase to provide the purified HBD or HBD-inducing polypeptide (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

5. Screening Assays

There are a variety of assays available for determining whether a candidate agent is a BD agent or a BD-inducing agent. In certain embodiment, the screening assays can be disposed in high-throughput formats. While not wishing to be bound by a particular theory, a BD-inducing agent may induce expression of BD, such as for example BD-2 and/or BD-3, in a host. In certain further aspects, the application provides pharmaceutical compositions comprising the agents identified in such assays together with a pharmaceutically acceptable xcipient.

In certain other aspects, the application provides assays fr identifying agents that may promote or potentiate the binding between a BD polypeptide and a protein such as CXCR4. In certain further aspects, the application provides pharmaceutical compositions comprising the agents identified in such assays together with a pharmaceutically acceptable xcipient.

The invention employs binding assays as screening methods. In certain embodiments, the screening of compounds that promote the binding of a BD to a CXCR4 is provided. In certain embodiment, portions of a BD and/or a CXCR4 sufficient to create the binding interface are employed in the binding assays.

The polypeptides involved in the binding assays may be either free in solution, fixed to a support, or expressed in a cell. A polypeptide involved in the binding may be labeled, thereby permitting determining amount of binding or lack thereof in the presence or absence of candidate agents to be screened. Competitive binding assays can be performed in which one of the polypeptides included in the assay is labeled. Conventional methods may be employed to decrease the chance that the labeling will interfere with the binding moiety's function. One may measure the amount of free label versus bound label to determine binding or promotion of binding.

In addition to cell-free assays, compounds can also be tested in cell-based assays, such as for example cell-based binding assays and reporter gene assays.

Various cell lines can be utilized for screening of the candidate agents, e.g., normal human oral epithelial cells. Candidate agents can be screened for their ability to induce in the cells expression of a BD such as for example a BD-2 and/or BD-3. Expression of a BD can be determined and measured by conventional methods such as for example northern blotting to determine mRNA level of the BD or western blotting to determine protein level of the BD. The BD expressed may be endogenous to the cells utilized in the assays, for example the normal human oral epithelial cells. As is known in the art, cell lines can also be created via transfections with nucleic acids encoding the proteins desired to be present for a subject assay, for example a BD-2 and/or a BD-3. Various reporter constructs may be used in accord with the methods of the invention and include, for example, reporter genes which produce such detectable signals as selected from the group consisting of an enzymatic signal, a fluorescent signal, a phosphorescent signal and drug resistance.

The disclosure further provides methods for identifying variants and fragments of a BD-inducing polypeptide of the invention. To illustrate, cells may be transfected with a BD-2 or BD-3 reporter construct, wherein a BD-inducing polypeptide responsive regulatory element of a BD-2 or BD-3 gene is operably linked to a reporter gene, and preferably a reporter gene that produces a fluorescent protein (e.g. green fluorescent protein) or an enzyme that can generate a fluorescent substrate or other detectable signal. The cells are then contacted with the BD-inducing polypeptide and reporter gene expression is assessed. In certain embodiments, an assay may comprise employing a cell that naturally has inducible BD-2 or BD-3 expression, such as a normal human oral epithelial cell. The cell may be transfected with a reporter construct or the expression of normal BD-2 or BD-3 transcript or polypeptide may be assessed.

Many useful pharmacological compounds are compounds structurally related to compounds that interact naturally with the targets, which may be a FAD-I, a viral envelope protein (such as for example a gp120 or gp41), a BD-2, a BD-3, CXCR4, or the binding interface between a BD and a CXCR4. Creating and examining the action of such molecules is known as “rational drug design,” and include making predictions relating to the structure of the targets. Thus, it is understood that a subject agent identified by the present invention may be a small molecule or any other compound (e.g., polypeptide or polynucleotide) that may be designed through rational drug design starting from known binders of the targets.

The goal of rational drug design is to produce structural analogs of biologically active target compounds. By creating such analogs, it is possible to fashion drugs that are more active or stable than the natural molecules, have different susceptibility to alteration or may affect the function of various other molecules. In one approach, one can generate a three-dimensional structure for molecules like the targets, and then design a molecule for its ability to interact with the targets. This could be accomplished by X-ray crystallography, computer modeling, or by a combination of both approaches.

6. BD and BD-Inducing Compositions

In certain aspects, the application provides compositions comprising an BD or BD-inducing agent and an excipient. Such compositions may be designed for delivery systemically or locally, and may be formulated for administration in any convenient way for use in human or veterinary medicine. In certain embodiments, the defensin-stimulating composition is formulated for local delivery to a particular epithelium, optionally a mucosal epithelium. For example, a composition may be formulated for delivery to the mouth, the eye, the skin, the vagina, the rectum, the intestines and the nose or other airways. In certain embodiments, the application provides methods for making a medicament comprising an BD or BD-inducing agent and an excipient for the administration by one of the above-described modes.

Thus, another aspect of the present invention provides compositions, optionally pharmaceutically acceptable compositions, comprising an amount, optionally a therapeutically-effective amount, of one or more of the agents or compositions described above, formulated together with one or more excipients, including additives and/or diluents. As described in detail below, the compositions may be specially formulated for administration in solid or liquid form, including those adapted for the following: (1) systemic or local oral administration, for example, drenches (aqueous or non-aqueous solutions or suspensions), tablets, boluses, powders, granules, pastes for application to the tongue, toothpastes or mouthwashes, films or strips (e.g., Listerine PocketPaks® Strip, which is a micro-thin starch-based film impregnated with ingredients); (2) parenteral administration, for example, by subcutaneous, intramuscular or intravenous injection as, for example, a sterile solution or suspension; (3) topical application, for example, as a cream, ointment or spray applied to the skin or mucous membrane; or (4) intravaginally or intrarectally, for example, as a pessary, cream, foam, or film that dissolves (e.g., the type of film used in vaginal contraceptive Films). However, in certain embodiments the subject BD or BD-inducing polypeptides or compositions may be simply dissolved or suspended in sterile water.

The phrase “therapeutically-effective amount” as used herein means that amount of a compound, material, or composition comprising an agent or composition of the present invention which is effective for producing some desired therapeutic effect by, for example, increasing BD (BD-2 or BD-3) production or inhibiting HIV entry in at least a sub-population of cells in an animal.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “excipient” as used herein means a material, composition or vehicle, such as a liquid or solid filler, diluent, excipient, solvent or encapsulating material, optionally pharmaceutically-acceptable, involved in administering the subject BD or BD-inducing polypeptide. Each excipient should be compatible with the other ingredients of the formulation and not injurious to the subject. Some examples of materials which can serve as pharmaceutically-acceptable excipients include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Compositions may also include excipients that are salts, preferably relatively non-toxic, inorganic and organic acid salts. These salts can be prepared in situ during the final isolation and purification of the agents or compositions of the disclosure, or by separately reacting a purified agent or composition with a suitable organic or inorganic acid, and isolating the salt thus formed. Representative salts include the chloride, hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19). Other salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the agents or compositions of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al. supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like: (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent or composition of the present invention with the carrier and, optionally, one or more accessory ingredients. In general, the formulations are prepared by uniformly and intimately bringing into association an agent or composition of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as tooth pastes or mouth washes and the like, each containing a predetermined amount of an agent or composition of the present invention as an active ingredient. An agent or composition of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the agents or compositions of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds (e.g., agents or compositions of the invention), may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more agents or compositions of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the FAD-I polypeptide.

Formulations of the present invention which are suitable for vaginal administration also include pessaries, tampons, creams, gels, pastes, foams or spray formulations containing such carriers as are known in the art to be appropriate.

Dosage forms for the topical or transdermal (systemic) or dermal (local) administration of an agent or composition of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent or composition of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Ophthalmic formulations, eye drops, ointments, powders, solutions and the like, are also contemplated as being within the scope of this invention.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the agents or compositions of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”. W.H. Freedman and CO., San Francisco, U.S.A., 1969 or “Livestock Feeds and Feeding” 0 and B books. Corvallis, Oreg., U.S.A., 1977).

BD or BD-inducing polypeptide may be incorporated into contraceptives, such as condoms, female condoms, spermicidal ointment, contraceptive films or sponges and the like.

In yet another embodiment, the BD or BD-inducing agent can be administered as part of a combinatorial therapy with other agents. For example, the combinatorial therapy can include a BD or BD-inducing agent with at least one antibacterial, antiviral or antifungal agent. A combinatorial therapy may include a BD or BD-inducing agent and a chemotherapeutic agent, such as cytosine, arabinoside, 5-fluorouracil, hydroxyurea, and methotrexate. In a preferred embodiment, a BD or BD-inducing agent is administered with one or more additional antiviral agents such as: a reverse transcriptase inhibitor, such as a nucleoside analog, e.g. Zidovudine, Didanosine, Zalcitabine, Stavudine, Lamivudine, and non-nucleoside analogs, e.g. Nevirapine, Delavirdine, or a protease inhibitor such as Saquinavir, Ritonavir, Indinavir and Nelfinavir. Others will be, in view of this disclosure, known to those of skill in the art.

7. Methods for Using Defensin-Stimulating Compositions

In certain aspects, the application relates to method for inhibiting an infection by HIV or a related virus in a subject, the method comprising administering to the subject an effective amount of an agent selected from the group consisting of: a BD agent; and a BD-inducing agent.

In certain aspects the application relates to a method for inhibiting the contraction of an HIV infection in a subject, the method comprising administering to the subject an effective amount of an agent selected from the group consisting of: an BD agent; and a BD-inducing agent.

In certain aspects the application relates to a method for inhibiting HIV entry into a cell, the method comprising contacting the cell with an effective amount of an agent selected from the group consisting of: a BD agent; and a BD-inducing agent.

Agents described herein may be used for HIV infections as well as infections of other viruses, and particularly those that associate with the CXCR4 receptor, such as the X4 types of HIV.

Recent studies also showed the presence of CXCR4 and other chemokine receptors in vascular smooth muscle cells, and may be involved in atherosclerosis [Schecter et al., Chemokine Receptors in Vascular Smooth Muscle, Microcirculation, June 2003, 10:265-72]. Thus, the BD or BD-inducing agents of the present invention may be used in treating atherosclerosis associated with CXCR4 in vascular smooth muscle.

Unlike most antiviral agents, resistance to beta-defensins is rare in pathogenic organisms. Accordingly, BD and BD-inducing agents may be used in situations where use of a traditional antiviral agent would be ill-advised because of the risk of resistance development. For example, BD and BD-inducing agents may be administered to patients that are unlikely to follow a complex dosing regimen or who do not have regular access to medical professionals.

8. Effective Dose

Toxicity and therapeutic efficacy of agents and compositions of the invention can be determined by standard pharmaceutical procedures in cell cultures or experimental animals, e.g., for determining the Ld50 (the dose lethal to 50% of the population) and the Ed50 (the dose therapeutically effective in 50% of the population). The dose ratio between toxic and therapeutic effects is the therapeutic index and it can be expressed as the ratio LD50/ED50. Agents or compositions which exhibit large therapeutic induces are preferred. While agents or compositions that exhibit toxic side effects may be used, care should be taken to design a delivery system that targets such agents or compositions to the site of affected tissue in order to minimize potential damage to uninfected cells and, thereby, reduce side effects.

The data obtained from the cell culture assays and animal studies can be used in formulating a range of dosage for use in humans. The dosage of such agents or compositions lies preferably within a range of circulating concentrations that include the ED50 with little or no toxicity. The dosage may vary within this range depending upon the dosage form employed and the route of administration utilized. For any agent or composition used in the method of the invention, the therapeutically effective dose can be estimated initially from cell culture assays. A dose may be formulated in animal models to achieve a circulating plasma concentration range that includes the IC50 (i.e., the concentration of the test agent or composition which achieves a half-maximal inhibition of symptoms) as determined in cell culture. Such information can be used to more accurately determine useful doses in humans. Levels in plasma may be measured, for example, by high performance liquid chromatography.

The practice of the present invention may employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Exemplification

The invention now being generally described, it will be more readily understood by reference to the following examples, which are included merely for purposes of illustration of certain aspects and embodiments of the present invention, and are not intended to limit the invention.

For reasons poorly understood, transmission of HIV-1 through oral secretions is uncommon [Rogers et al.; Moore et al.]. Despite the ready demonstration of HIV-1 RNA, proviral DNA and infected cells in salivary secretions of infected persons [Goto, et al.; Baron et al.], infectious virus is rarely isolated from saliva [Rogers et al.; Barr et al.; Coppenhaver, et al.]. Thus, diminished infectivity of HIV-1 within oropharyngeal tissues [Herz et al.] may underlie the infrequent transmission of HIV-1 through this route. A better understanding of this apparent protection is particularly important as more than 90% of HIV-1 cases worldwide have been transmitted across other mucosal surfaces [Smith et al.].

Numerous studies have been conducted to identify the HIV inhibitory activity in saliva of healthy and infected individuals [reviewed by Shugars et al.]. Many salivary inhibitors of HIV-1 have been proposed; e.g., amylase, lactoferrin, proline-rich peptides, salivary mucins, thrombospondin, and secretory leukocyte protease inhibitor [Id.]. The importance of these agents in oral mucosal protection remains to be demonstrated. It is shown here that mRNA expression of beta-defensins can be induced in oral epithelial cells by exposure to HIV-1 and that these defensins can inhibit HIV propagation in vitro. In contrast to other mucosal body sites, where hBD-2 and -3 are induced only during inflammation [O'Neil et al.; Wehkamp et al; Bajaj-Elliott et al.; Ong et al.; Liu et al.], expression of these host defense agents is always measurable even in normal uninflamed oral epithelium [Dale et al.]. Since the reported concentration of hBD-2 in normal oral epithelium is about 10 μmoles/g tissue [Sawaki et al.], well within the inhibitory concentrations reported in the in vitro experiments with the X4 phenotype, further induction of hBD-2 (or hBD-3) by mucosal exposure to virus may provide protection against X4-tropic and potentially also against R5-tropic viruses. It should be noted that induction of hBD mRNA expression in NHOEC is accompanied by increased expression of hBD protein. (A. Weinberg et al., unpublished data).

As shown in the examples and figures herein, HIV-1 induced expression of hBD-2 and -3 mRNA in normal human oral epithelium and cells (“NHOEC”) and that these defensins, but not hBD-1, inhibit HIV-1 replication in immunocompetent cells. Inhibition involves the binding of HIV-1 directly, as well as an additional downmodulation of cell surface CXCR4 expression. Inhibition of HIV-1 replication by beta-defensins may play an important role in protecting the oral cavity and other mucosal surfaces from infection; preferential inhibition of CXCR4-tropic (X4) HIV-1 strains may help to explain the selective acquisition of CCR5-tropic (R5) HIV-1 isolates after in vivo mucosal exposure.

While it is premature to speculate how beta-defensins bind HIV-1, an interaction with gp120 is plausible. Polyanionic compounds exert their anti-HIV-1 activity by binding to the positively charged sites in the V3 loop of gp120 [Schols et al.; Witvrouw et al.]. Like other polycationic peptides which block infection with X4 HIV-1 isolates (e.g., T22, T134, and ALX40-4C) [De Clercq et al.], the direct antiretroviral effect of beta-defensins might be predicted to be very different, perhaps interacting with other viral surface domains.

### EXAMPLE 1

### Materials and Methods

Cells and Viruses

Peripheral blood mononuclear cells (PBMC) were stimulated with phytohemagglutinin (PHA) and interleukin (IL)-2 [Pauwels et al.]. MT-4 and CEM X4/R5 T-cell lines, and GHOST CXCR4 and CCR5-transfected osteosarcoma cells cotransfected with the HIV-2 long terminal repeat driving expression of the green fluorescent protein (hGFP), and all viral isolates were obtained through the AIDS Research and Reference Reagent Program. Normal human oral epithelium and cells (NHOEC) were prepared as described [Krisanaprakorkit et al., 1998 and 2000]. Viral stocks were propagated in PHA-stimulated, IL-2 treated PBMC, and tissue culture dose for 50% infectivity was determined [Quinones-Mateu et al.].

Generation of Recombinant Human β-Defensins (hBDs)

Recombinant hBD-1 and -2 (rhBD-1 and -2) were produced from the infection of Sf21 cells with baculovirus constructs as described [Valore et al.]. Recombinant hBD-3 (rhBD-3) was produced using an hBD-3-His tag fusion construct, generated by PCR and cloned into pET-30c [Harder et al.]. Identity, purity and biological activity of rhBD-1, -2, and -3 were confirmed by acid urea-PAGE migration, Western analysis with native peptides, N-terminal amino acid sequencing, matrix assisted laser desorption ionization time of flight mass spectrometry, and killing of Escherichia coli ML35p [Valore et al.; Harder et al.].

Real-Time RT-PCR Assay to Quantify hBD mRNA

RNA was extracted from NHOEC monolayers post HIV-1 challenge [multiplicity of infection (MOI), 0.01 infectious unit/cell] using TRIzol according to the manufacturer's protocol (Invitrogen Life Technologies, Carlsbad, Calif., USA). Human keratin 5 RNA was used to normalize RNA content in each preparation. Intron spanning primers used and PCR conditions for these reactions have been described previously [Krisanaprakorkit et al., 1998 and 2000; Harder et al.]. Each 25-μl PCR mixture consisted of 125 ng RNA, primers (0.4 μM each), 0.4 mM dNTPs, 5 mM MgCl2, a mixture of reverse transcriptase and Taq DNA polymerase, 13 PCR buffer, RNase inhibitor (5 U), and SYBR Green dye diluted 1:2500 (Sigma, St. Louis, Mo., USA) as described [Weber et al.]. Standard curves were constructed using RNA generated by transcribing hBD-1, -2, or -3 plasmids using the RiboProbe in vitro transcription system (Promega, Madison, Wis., USA). Concentration of mRNAs was determined by spectrometry at 260 nm. Single-stock solutions of serial dilutions from 107 to 10 RNA copies were prepared and stored at −80° C. All real-time RT-PCR amplifications, data acquisition, and analysis were performed using the Smart Cycler System, software version 1.2d (Cepheid, Sunnyvale, Calif., USA).

Anti-HIV-1 Activity and Cytotoxicity of hBD

HIV-1 isolates were incubated with increasing concentrations (5-40 μg/ml) of hBD-1, -2, and -3, in three different conditions: high salt complete medium (RPMI-1640 or DMEM supplemented with 10% fetal bovine serum, FBS); high salt medium in the absence of FBS; or low salt medium (10 mM phosphate buffer), 37° C. for 1 h. Respective mixtures were used to infect PBMC, Ghost X4/R5 or CEM X4/R5 cells at an MOI of 0.01 IU/ml. After 2 h incubation at 37° C., 5% CO₂, cells were washed three times with phosphate-buffered saline (PBS) and cultured in complete medium for 48 h. In the case of Ghost X4/R5 cells, these were washed, resuspended in PBS, and analyzed by fluorescence microscopy for GFP expression as described [Morner et al.]. Cell-free supernatants from PBMC, Ghost X4/R5 and CEM X4/R5 cultures were used to monitor infectivity by the reverse transcriptase (RT) assay [Quinones-Mateu et al.]. The 50% inhibitory concentration (IC50) of each hBD was determined using X4 or R5 HIV-1 isolates. Viruses (0.01 MOI) were incubated with increasing concentrations of hBD-1, -2, and 3 (up to 40 g/ml) in low salt medium for 1 h and used to infect CEM X4/R5 cells. After 2 h incubation at 37° C., 5% CO₂, cells were washed twice with PBS and cultured in complete medium. Supernatant samples were removed on day 5 post-infection and virus production was measured using AIDS 2003, Vol 17 No X 2 the RT assay [Id.]. Cytotoxicity of hBD was quantified by determining the number of viable cells using a tetrazolium-based colorimetric (MTT) assay [Pauwels et al.].

Flow Cytometric Analysis

Unstimulated PBMC were treated with 30 μg/ml hBD-1, -2, or -3 in high salt medium (RPMI-1640) in the absence of FBS, 3 h. The CXCR4 natural ligand SDF-1α (R&D Systems, Minneapolis, Minn., USA) and the CCR5 antagonist PSC-RANTES were used as controls. Cells were incubated with peridinin chlorophyll protein-conjugated anti-human CD4 antibody, and either phycoerythrin (PE)-conjugated anti-human CXCR4 antibody, PE-conjugated anti-human CCR5 antibody, or PE-conjugated mouse immunoglobulin G2a (IgG2a), isotype standard (PharmMingen, San Diego, Calif., USA) [Salkowitz et al.]. Fluorescence intensity was reported as receptor density by quantitative flow cytometry (FACSCaliber; Becton Dickenson, San Jose, Calif., USA) [Iyer et al.]. Data were analyzed using CELLQuest software (Becton Dickenson).

Confocal Microscopy

CEM X4/R5 cells were grown in RPMI-1640 medium containing 5% FBS and 400 μg/ml G418. Cells were collected, washed twice with PBS, resuspended in RPMI with 0.5% FBS or supplemented with 20 μg/ml recombinant hBD-2 or -3, and incubated at 37° C., 5% CO₂ for 3 h. A second aliquot of cells, after incubation with hBD, was treated with FACS/Perm (PharMingen) at room temperature for 10 min and then washed three times with PBS. A third aliquot of cells was fixed in 1% paraformaldehyde on ice for 30 min, washed three times with PBS, and then incubated with 20 μg/ml hBD-2 or -3. Cells were stained with PE-labeled CXCR4 or CCR5 (PharMingen), or with primary goat anti-hBD-2 antibodies (Cell Sciences, Norwood, Mass., USA), or with rabbit anti-hBD-3 antibodies (Orbingen, San Diego, Calif., USA) at room temperature for 90 min, followed by washing three times with PBS. Fluorescein isothiocyanate-labeled rabbit anti-goat IgG (Jackson ImmunoResearch, West Grove, Pa., USA) for detection of hBD-2, or goat anti-rabbit IgG (Sigma) for detection of hBD-3, were added, respectively, at room temperature and incubated for 90 min. Cells were washed twice with PBS, resuspended in 1% paraformaldehyde (except for the pre-fixed cells), and stored at 48C prior to analysis. All samples were observed using a dual scanning confocal microscope system (Zeiss LSM 510, Oberkochem, Germany) and analyzed with the Zeiss LSM 5 Image Browser.

Immunogold Transmission Electron Microscopy

MT4 cells in RPMI, or X4 strain B-HXB2 viral particles in 10 mM phosphate buffer (PB), were incubated with 20 μg/ml hBD-2 and -3 at 37° C. for 1 h. Cells and virions were centrifuged (15 min, 1200 rpm for cells; 30 min, 35000 3 g for virus) and washed twice with PBS to remove unbound hBD. Cells and virions were mixed and fixed with 4% paraformaldehyde/0.5% glutaraldehyde, dehydrated, embedded in LR WHITE resin (London Resin Company Ltd, Berkshire, UK), and labeled after embedding as described [Briquet et al.]. Ultrathin sections were incubated with primary rabbit anti-hBD-2 or anti-hBD-3 antibody (1:100 dilution) overnight at 48C, washed, and incubated for 2 h at room temperature with a 1:10 dilution of goat anti-rabbit IgG conjugated with 10 nm gold particles (Ted Pella Inc., Redding, Calif., USA) as the second antibody. Negative controls included HIV-infected cells incubated with both hBD and only the secondary gold-conjugated antibody, or HUV-infected cells incubated with primary and secondary antibodies in the absence of hBD. Embedding and preparation for conventional transmission electron microscopy were performed as described [Id.].

### EXAMPLE 2

### HIV-1 Induces hBD-2 and hBD-3 but Not hBD-1 mRNA in NHOEC

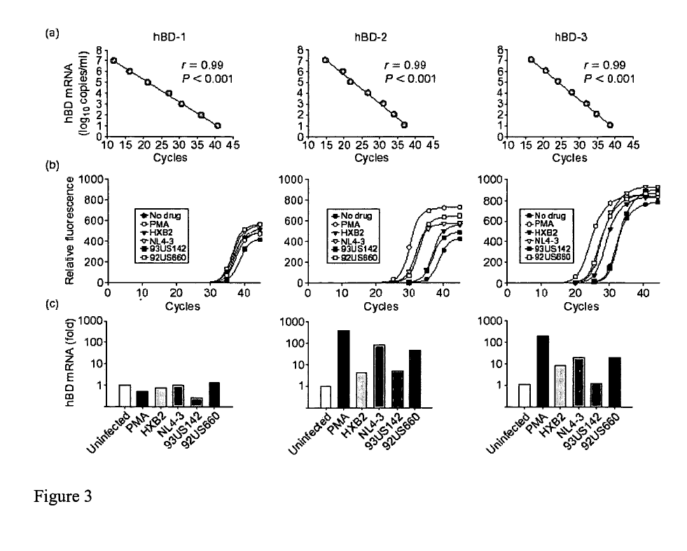

NHOEC monolayers were challenged with four different HIV-1 strains representing both viral bio-phenotypes (i.e., SI/X4, B-HXB2 and B-NL4-3; NSI/R5, B-93US142 and B-92US660). Forty-eight hours postinfection, hBD-1, -2, and -3 mRNA expression was measured by real-time PCR. All HIV-1 strains induced hBD-2 and hBD-3 mRNA 4- to 78-fold above baseline (FIG. 3). No induction of hBD-1 mRNA was observed. Supernatants from uninfected MT4 cells or PBMC, used to grow respective viral strains, did not induce either hBD-2 or -3 mRNA expression (data not shown). HBD-2 and -3 transcript expression increased with viral exposure time and was maintained as long as 72 h post-exposure (data not shown). Finally, although HIV-1 can infect epithelial cells from other mucosal surfaces [Yahi. et al.; Fotopoulos et al.], analyses of viral RT activity in culture supernatant [Quinones-Mateu et al.] and real-time PCR to detect proviral DNA in cells failed to detect infection of NHOEC by HIV-1 (data not shown).

### EXAMPLE 3

### HBD-2 and hBD-3 Inhibit HIV-1 Replication

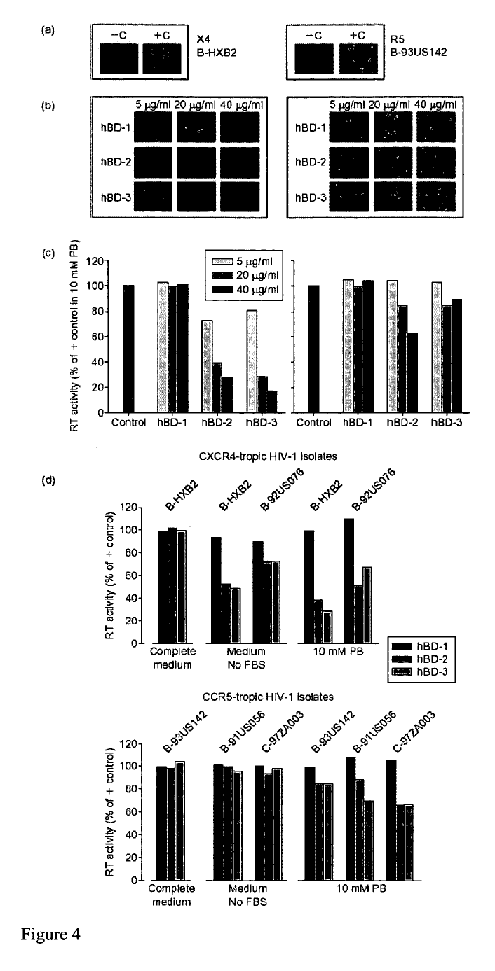

Since the antibacterial activity of beta-defensins is sensitive to high salt and serum concentrations, the anti-HIV-1 activity of hBD was initially evaluated in a low salt, serum free environment, mimicking oral mucosal conditions [Mandel et al.]. Two HIV-1 isolates (X4 B-HXB2 and R5 B-93US142) were preincubated for 1 h with increasing concentrations of recombinant hBD-1, -2, and -3, in 10 mM phosphate buffer (PB). GHOST CCR5/CXCR4 cells were then exposed to the mixtures for 48 h in complete medium. While hBD-1 had no effect, preincubation of HIV-1 with either hBD-2 or hBD-3 in 10 mM PB showed anti-HIV-1 activity (FIG. 4b), which was concentration dependent and greater against the X4 B-HXB2 strain than against the R5 B-93US142 isolate (61% versus 15% inhibition with 20 μg/ml hBD-2, respectively) (FIG. 4c). When CXCR4- and CCR5-tropic HIV-1 strains were preincubated with beta-defensins in high salt medium (DMEM) supplemented with 10% FBS, no antiviral effect was detected (FIG. 4d). However, preincubation in DMEM without FBS inhibited replication of X4, but not R5, HIV-1 isolates (FIG. 4d). Under low salt conditions (i.e., 10 mM PB, no FBS) the 50% inhibitory concentration for both agents against X4 and R5 viruses ranged from 9 to 19 μg/ml and 20 to 40 μg/ml, respectively. These findings suggest that hBD-2 and -3 may have a direct electrostatic interaction with HIV-1 particles that inhibits infection. In addition, the greater activity against X4 HIV-1 strains suggested either an electrostatic preference for X4 versus R5 binding and/or a selective effect on the viral co-receptor.

### EXAMPLE 4

### HBDs Are Not Toxic to Human Cells

A thiazolyl blue-based colorimetric assay (MTT method) [Pauwels et al.] revealed no cytotoxicity against PBMC, CEM X4/R5, MT4 or GHOST X4/R5 cells using up to 40 μg/ml of each hBD, in the presence or absence of serum.

### EXAMPLE 5

### HBD-2 and -3 Downmodulate CXCR4, but Not CCR5

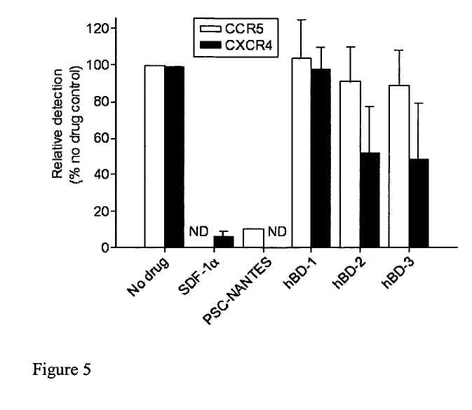

The more effective inhibition of X4 HIV-1 strains over R5 HIV-1 isolates (FIG. 4), led us to ask whether hBD-2 and -3 interact with the HIV co-receptor CXCR4. Flow cytometric analysis of hBD-1, -2 or -3 preincubated PBMC showed that CCR5 expression was not altered by hBD (FIG. 5). Incubation with hBD-1 did not affect surface expression of CXCR4. Surface expression of CXCR4 was decreased by 51%±18% and 52%±20% (SD) respectively after incubation with 30 μg/ml of hBD-2 or hBD-3 (FIG. 5). Similar results were obtained with CEM cells expressing CXCR4 and CCR5.

To explore the mechanism of this effect, CEM X4/R5 cells were incubated with hBD-2 and -3 and then examined for surface expression of CXCR4 and CCR5 by confocal microscopy. This exposure dramatically decreased surface expression of CXCR4 (but not CCR5). Subsequent labeling with polyclonal antibodies against hBD-2 or hBD-3 failed to detect these peptides on the cell surface. Since chemokine receptors may internalize after ligation, CEM X4/R5 cells were first fixed with paraformaldehyde, then incubated with hBD-2 and finally labeled with anti-hBD-2 antibody. This time, hBD-2 was found bound to the cell membrane. Finally, to visualize hBD-2 internalization, live CEM X4/R5 cells were incubated first with hBD-2, then permeabilized and incubated with anti-hBD-2 antibodies. HBD-2 was identified by confocal microscopy with a staining pattern suggestive of internalization. Similar results were observed when CEM X4/R5 cells were treated with hBD-3. Collectively, these results suggest that both hBD-2 and -3 bind to cell surface CXCR4 and induce internalization of the bound complex.

### EXAMPLE 6

### HBD-2 and -3 Interact with Both HIV-1 and the Host Cell

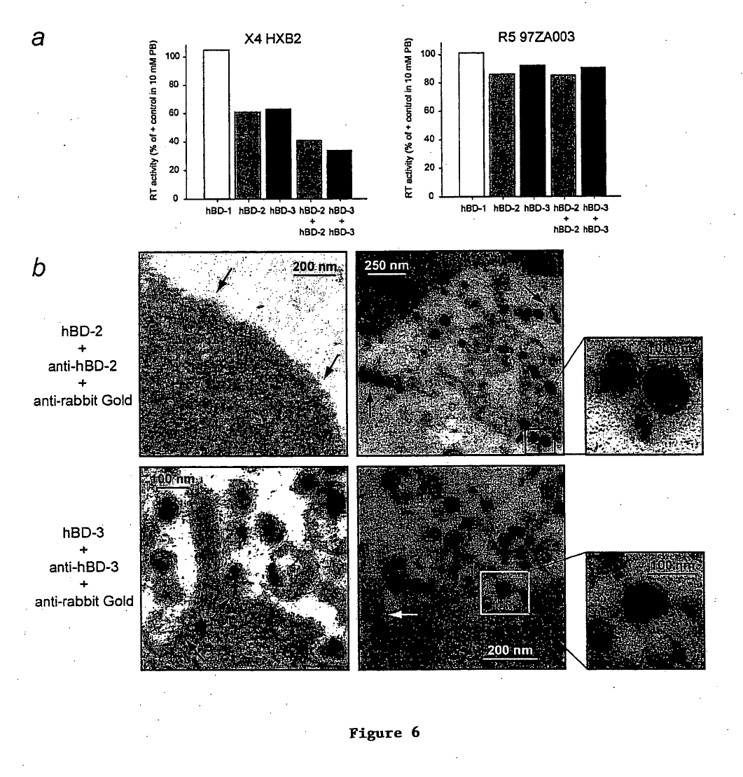

In order to verify a direct hBD-virion interaction, the X4 B-HXB2 and R5-C-97ZA003 HIV-1 strains was incubated with 20 μg/ml of each hBD in 10 mM PB for 1 h, followed by pelleting and extensive washing. Virions were then used to infect GHOST X4/R5 cells. The anti-HIV-1 effect of hBD-2 and -3 was maintained after washing, suggesting a direct and irreversible effect on the virion. Moreover, subsequent addition of hBD-2 or hBD-3 to the cell-virus mixture enhanced anti-HIV-1 activity only against the CXCR4-tropic BHXB2 strain (FIG. 6a). Taken together, these data suggest that the inducible defensins have both a direct inhibitory effect on HIV-1 infectiousness and an additional antiviral effect that is probably mediated through downmodulation of CXCR4. To further define these interactions, MT4 cells, infected with the CXCR4 tropic B-HXB2, were incubated with hBD-2 or -3 in RPMI, followed by the addition of anti-hBD-2 or -3 antibodies and goat anti-rabbit IgG conjugated with 10-nm gold particles. Gold particles were observed bound both to virions and to the MT4 cellular membrane in samples incubated with hBD-2 or -3 (FIG. 6b), but not in samples incubated in the absence of hBD. Taken together, these results indicate that hBD-2 and -3 bind directly to virions inducing irreversible inhibition of HIV replication and also bind to host cells inducing downmodulation of the CXCR4 chemokine coreceptor.

### REFERENCES

1. Krisanaprakornkit S, Weinberg A, Perez C N, Dale B A. Expression of the peptide antibiotic human beta-defensin 1 in cultured gingival epithelial cells and gingival tissue. Infect Immun 1998, 66:4222-4228.

2. Krisanaprakornkit S, Kimball JR, Weinberg A, Darveau R P, Bainbridge B W, Dale B A. Inducible expression of human betadefensin 2 by Fusobacterium nucleatum in oral epithelial cells: multiple signaling pathways and role of commensal bacteria in innate immunity and the epithelial barrier. Infect Immun 2000, 68:2907-2915.

3. Pauwels R, Balzarini J, Baba M, et al. Rapid and automated tetrazolium-based colorimetric assay for the detection of anti-HIV compounds. J Virological Methods 1988, 20:309-321.

4. Quinones-Mateu M E, Ball S C, Marozsan A J, et al. A dual infection/competition assay shows a correlation between ex vivo human immunode.ciency virus type 1 .tness and disease progression. J Virol 2000, 74:9222-9233.

5. Valore E V, Park C H, Quayle A J, Wiles K R, McCray P B, Jr., Ganz T. Human beta-defensin-1: an antimicrobial peptide of urogenital tissues. J Clin Invest 1998, 101:1633-1642.

6. Harder J, Bartels J, Christophers E, Schroder J M. Isolation and characterization of human beta-defensin-3, a novel human inducible peptide antibiotic. J Biol Chem 2001, 276:5707-5713.

7. Weber J, Rangel H R, Chakraborty B, et al. A novel TaqMan realtime PCR assay to estimate ex vivo human immunodeficiency β-defensins 2 and 3 inhibit HIV-1 Quinones-Mateu et al. 9 virus type 1 fitness in the era of multi-target (pol and env) antiretroviral therapy. J Gen Virol 2003, 84:2217-2228.

8. Morner A, Bjorndal A, Albert J, et al. Primary human immunodeficiency virus type 2 (HIV-2) isolates, like HIV-1 isolates, frequently use CCR5 but show promiscuity in coreceptor usage. J Virol 1999, 73:2343-2349.

9. Salkowitz J R, Purvis S F, Meyerson H, et al. Characterization of high-risk HIV-1 seronegative hemophiliacs. Clin Immunol 2001, 98:200-211.

10. Iyer S B, Hultin L E, Zawadzki J A, Davis K A, Giorgi J V. Quantitation of CD38 expression using QuantiBRITE beads. Cytometry 1998, 33:206-212.

11. Briquet S, Vaquero C. Immunolocalization studies of an antisense protein in HIV-1-infected cells and viral particles. Virology 2002, 292:177-184.

12. Yahi N, Baghdiguian S, Moreau H, Fantini J. Galactosyl ceramide (or a closely related molecule) is the receptor for human immunodeficiency virus type 1 on human colon epithelial HT29 cells. J Virol 1992, 66:4848-4854.

13. Fotopoulos G, Harari A, Michetti P, Trono D, Pantaleo G, Kraehenbubl J P. Transepithelial transport of HIV-1 by M cells is receptor-mediated. Proc Natl Acad Sci USA 2002, 99:9410-9414.

14. Mandel I D. Saliva. St. Louis: C. V. Mosby Co, 1972.

15. Rogers M F, White C R, Sanders R, et al. Lack of transmission of human immunodeficiency virus from infected children to their household contacts. Pediatrics 1990, 85:210-214.

16. Moore B E, Flaitz C M, Coppenhaver D H, Nichols M, Kalmaz G D, Bessman J D, et al. HIV recovery from saliva before and after dental treatment: inhibitors may have critical role in viral inactivation. J Am Dental Assoc 1993, 124:67-74.

17. Goto Y, Yeh C K, Notkins A L, Prabhakar B S. Detection of proviral sequences in saliva of patients infected with human immunodeficiency virus type 1. AIDS Res Hum Retroviruses 1991, 7:343-347.

18. Baron S, Poast J, Cloyd M W. Why is HIV rarely transmitted by oral secretions? Saliva can disrupt orally shed, infected leukocytes. Arch Intern Med 1999, 159:303-310.

19. Barr C E, Miller L K, Lopez M R, et al. Recovery of infectious HIV-1 from whole saliva. J Am Dental Assoc 1992, 123:36-37.

20. Coppenhaver D H, Sriyuktasuth-Woo P, Baron S, Barr C E, Qureshi M N. Correlation of nonspeci.c antiviral activity with the ability to isolate infectious HIV-1 from saliva. New Engl J Med 1994, 330:1314-1315.

21. Herz A M, Robertson M N, Lynch J B, Schmidt A, Rabin M, Sherbert C, et al. Viral dynamics of early HIV infection in neonatal macaques after oral exposure to HIV-2287: an animal model with implications for maternal-neonatal HIV transmission. J Med Primatol 2002, 31:29-39.

22. Smith P D, Li L, Meng G. Mucosal events in the pathogenesis of human immunodeficiency virus type 1 infection. J Infect Dis 1999, 179 Suppl 3:S436-440.

23. Shugars D C, Wahl S M. The role of the oral environment in HIV-1 transmission. J Am Dental Assoc 1998, 129:851-858.

24. O'Neil D A, Porter E M, Elewaut D, et al. Expression and regulation of the human beta-defensins hBD-1 and hBD-2 in intestinal epithelium. J Immunol 1999, 163:6718-6724.

25. Wehkamp J, Fellermann K, Herrlinger K R, et al. Human betadefensin 2 but not beta-defensin 1 is expressed preferentially in colonic mucosa of in.ammatory bowel disease. Eur J Gastroenterol Hepatol 2002, 14:745-752.

26. Bajaj-Elliott M, Fedeli P, Smith G V, Domizio P, Maher L, Ali R S, et al. Modulation of host antimicrobial peptide (beta-defensins 1 and 2) expression during gastritis. Gut 2002, 51:356-361.

27. Ong P Y, Ohtake T, Brandt C, Strickland I, Boguniewicz M, Ganz T, et al. Endogenous antimicrobial peptides and skin infections in atopic dermatitis. [see comments] New Engl J Med 2002, 347:1151-1160.

28. Liu L, Wang L, Jia H P, Zhao C, Heng H H Q, Schutte B C, et al. Structure and mapping of the human beta-defensin HBD-2 gene and its expression at sites of in.ammation. Gene 1998, 222:237-244.

29. Dale B A, Kimball J R, Krisanaprakornkit S, Roberts F, Robinovitch M, O'Neal R, Localized antimicrobial peptide expression in human gingiva. J Periodontal Res 2001, 36:285-294.

30. Sawaki K, Mizukawa N, Yamaai T, Yoshimoto T, Nakano M, Sugahara T. High concentration of beta-defensin-2 in oral squamous cell carcinoma. Anticancer Res 2002, 22:2103-2107.

31. Schols D, Pauwels R, Desmyter J, De Clercq E. Dextran sulfate and other polyanionic anti-HIV compounds specifically interact with the viral gp120 glycoprotein expressed by T-cells persistently infected with HIV-1. Virology 1990,175:556-561.

32. Witvrouw M, Este J A, Quinones-Mateu M E, Reymen D, Andrei G, Snoeck R, et al. Activity of a sulfated polysaccharide extracted from the red seaweed Aghardhiella tenera against human immunodificiency virus and other enveloped viruses. Antiviral Chem Chemother 1994, 5:297-303.

33. De Clercq E. New developments in anti-HIV chemotherapy. Biochim Biophys Acta 2002, 1587:258-275.

Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

I claim:

1. A method for inhibiting HIV infection in a subject, comprising administering to the subject an effective amount of an agent selected from the group consisting of:
a. an Beta Defensin (BD) agent; and
b. an Beta Defensin-inducing agent.

2. A method for inhibiting the contraction of an HIV infection in a subject, comprising administering to the subject an effective amount of an agent selected from the group consisting of:
a. an BD agent; and
b. an BD-inducing agent.

3. A method for inhibiting HIV entry into a cell, the method comprising contacting the cell with an effective amount of an agent selected from the group consisting of:
a. an BD agent; and
b. an BD-inducing agent.

4. A method of any of claims 1-3, wherein the BD agent is a human Beta Defensin agent (HBD).

5. A method of claim 4, wherein said HBD agent is an human Beta defensin-2 agent (HBD2).

6. A method of claim 5, wherein said HBD-2 agent is a polypeptide comprising an amino acid sequence at least 90% identical to an amino acid sequence selected from the group consisting of: SEQ ID NO:1 and SEQ ID NO:2.

7. A method of claim 5, wherein the HBD agent is an HBD-2 agent, and wherein further said HBD-2 agent is a polypeptide encoded by a nucleic acid that is at least 90% identical to a nucleic acid selected from the group consisting of: SEQ ID NOs:4-7.

8. A method of claim 4, wherein said HBD agent is an human Beta defensin-3 agent (HBD3).

9. A method of claim 8, wherein said HBD-3 agent is a polypeptide comprising an amino acid sequence at least 90% identical to an amino acid sequence as set forth in SEQ ID NO:15.

10. A method of claim 9, wherein the HBD agent is an HBD-3 agent, and wherein further said HBD-3 agent is a polypeptide encoded by a nucleic acid that is at least 90% identical to a nucleic acid selected from the group consisting of: SEQ ID NOs: 16-18.

11. A method of any of claims 1-3, wherein the HBD-inducing agent is a polypeptide comprising an amino acid sequence at least 90% identical to an amino acid sequence selected from the group consisting of SEQ ID NOs:3, 9, 11, and 13.

12. A method of any of claims 1-3, wherein the HBD-inducing agent is a polypeptide encoded by a nucleic acid that is at least 90% identical to a nucleic acid having a nucleotide sequence selected from the group consisting of SEQ ID NOs:8, 10, 12, and 14.

13. A method of any of claims 1-3, wherein the HBD agent has a 50% effectiveness at a concentration of about 10 micromolar or less.

14. A method of any of claims 1-3, wherein the agent is administered systemically.

15. A method of claim 14, wherein the agent is administered directly to the bloodstream.

16. A method of any of claims 1-3, wherein the agent is administered locally.

17. A method of claim 16, wherein the agent is administered to a portion of the body selected from the group consisting of: the mouth, the nasopharyngeal tract, the anus, the vagina, the penis, the skin, and the eye.

18. A method of claim 16, wherein the agent is administered to a mucous membrane.

19. A method of any of claims 1-3, wherein the agent is administered in a form selected from the group consisting of: a mouthwash, a toothpaste, an aerosol, a rectal or vaginal suppository, a rectal or vaginal cream, a rectal or vaginal film, a skin lotion, a condom, an eye drop, and an eye ointment.

20. A method of any of claim 1-3, wherein the agent is administered in combination with an additional antiviral agent.

21. The method of claim 20, wherein the antiviral agent targets a portion of the HIV virus selected from the group consisting of: an HIV protease and an HIV reverse transcriptase.

22. The method of any of claims 1-3, wherein the HIV is an HIV that associates with CXCR4.

23. The method of any of claims 1-3, wherein the HIV is an X4-type HIV.

24. The method of any of claims 1-3, wherein the HBD-inducing agent induces expression of HBD-2, HBD-3, or both.

25. A method of identifying a BD-inducing agent comprising:
-providing cells capable of expressing a BD-polypeptide;
-contacting said cells with an agent;
-determining the level of expression of said BD-polypeptide in the presence of said agent and comparing the level of expression of said BD-polypeptide in the presence of said agent to the expression of said BD-polypeptide in the absence of said agent;
-wherein an increase in the expression of said BD-polypeptide in indicative of a BD-inducing agent.

26. The method of claim 25, wherein the BD-polypeptide is a human BD-polypeptide.

27. The method of claim 26, wherein said HBD polypeptide is an human Beta defensin-2 polypeptide.

28. The method of claim 27, wherein said HBD-2 polypeptide comprises an amino acid sequence at least 90% identical to an amino acid sequence selected from the group consisting of: SEQ ID NO:1 and SEQ ID NO:2.

29. The method of claim 27, wherein the HBD polypeptide is encoded by a nucleic acid that is at least 90% identical to a nucleic acid selected from the group consisting of: SEQ ID NOs:4-7.

30. The method of claim 26, wherein said HBD polypeptide is an human Beta defensin-3 polypeptide (HBD3).

31. The method of claim 30, wherein said HBD-3 agent is a polypeptide comprising an amino acid sequence at least 90% identical to an amino acid sequence as set forth in SEQ ID NO:15.

32. The method of claim 30, wherein the HBD agent is an HBD-3 agent, and wherein further said HBD-3 agent is a polypeptide encoded by a nucleic acid that is at least 90% identical to a nucleic acid selected from the group consisting of: SEQ ID NOs: 16-18.

33. The method of claim 25, further comprising the step of preparing a pharmaceutical composition comprising the agent identified to be a BD-inducing agent.

34. The method of claim 25, wherein said cells express endogenous BD-polypeptide.

35. A method of identifying agents that potentiate the interaction between a BD-polypeptide and a chemokine receptor, comprising
(i) providing a chemokine receptor and a BD-polypeptide;
(ii) adding an agent to step (i);
(iii) determining the interaction of said chemokine receptor and said BD-polypeptide in the presence of said agent to the interaction in the absence of said agent;
wherein an increase in the interaction of said BD-polypeptide with said chemokine receptor is indicative of a agent that potentiates the interaction between said BD-polypeptide and said chemokine receptor.

36. The method of claim 35, wherein said chemokine receptor is CXCR4.

37. The method of any one of claims 1-3, wherein the BD-inducing agent is selected from the group consisting of a small molecule, a polypeptide, a nucleic acid, and a peptidomimetic.

38. The method of claim 37, wherein said BD-inducing agent is a polypeptide having an amino acid sequence at least 90% identical to the amino acid sequence selected from the group consisting of SEQ ID NOs:3, 9, 11, and 13.

39. The method of claim 37, wherein said BD-inducing agent is a polypeptide encoded by a nucleic acid sequence at least 90% identical to the nucleotide sequence selected from the group consisting of SEQ ID NOs:8, 10, 12, and 14.
